(19) United States
(12) Patent Application Publication
(10) Pub. No.: US 2021/0259944 A1
(43) Pub. Date: Aug. 26, 2021

(54) PERSONAL CARE FORMULATIONS COMPRISING THICKENED ORGANIC LIQUIDS

(71) Applicant: NOURYON CHEMICALS INTERNATIONAL B.V., Arnhem (NL)

(72) Inventors: Shawn ZHU, Stormvilie, NJ (US); John Socrates THOMAIDES, Berkeley Heights, NJ (US); John M. MORALES, Warren, NJ (US); Kaaliyah WILSON, Dunellen, NJ (US); Kristin GOL.AS, Raritan, NJ (US); Qiwei HE, Belle Mead, NJ (US); Gary Theodore MARTSNO, Monmouth Junction, NJ (US)

(73) Assignee: NOURYON CHEMICALS iNTERNATIONAL B.V., Arnhem (NL)

(21) Appl. No.: 16/973,904

(22) PCT Filed: Jun. 11, 2019
(86) PCT No.: PCT/EP2019/065101

§ 371 Date: Dec. 10, 2020

Related U.S. Application Data
(60) Provisional application No. 62/684,455, filed on Jun. 13, 2018.

(30) Foreign Application Priority Data
Aug. 10, 2018 (EP) 18188395.0

(51) Int. Cl. A61K 8/81; A61K 8/06; A61Q 17/04; A61Q 19/00; A61Q 5/00

(52) U.S. Cl. CPC A61K 8/8152; A61K 8/064; A61K 8/062; A61Q 17/04; A61K 2800/594; A61Q 5/00; A61K 2800/33; A61K 2800/48; A61Q 19/00

(57) ABSTRACT

A personal care formulation comprises a dermatologically acceptable organic liquid and a cross linked (meth)acrylate copolymer, wherein the organic liquid is thickened by the cross linked (meth)acrylate copolymer, and wherein the cross linked (meth)acrylate copolymer comprises at least one alkyl (meth)acrylate and at least one of the following monomers: a bicyclic (meth)acrylate different from the alkyl (meth)acrylate, and an aromatic vinyl monomer. In one embodiment the formulation can further comprise water and be in the form of a water-in-oil emulsion. In another embodiment the formulation can comprise water and an aqueous thickener and be in the form of an oil-in-water emulsion. The personal care formulation can be a hair care formulation, a skin care formulation, or a sunscreen formulation.

### FIELD OF THE INVENTION

This application relates to personal care formulations comprising thickened organic liquids. More particularly this application relates to personal care formulations comprising thickened organic liquids wherein the oil thickener is a crosslinked polymer.

### BACKGROUND OF THE INVENTION

Many organic liquids (i.e., oils) are low viscosity liquids that tend to run-off during use. A thickened oil can reduce the oil run-off during use. In addition, a thickened oil has various applications. In personal care, a thickened oil can be used to provide emollient properties or water-proof a person's skin. One example is Johnson's® Baby Oil Gel (Johnson & Johnson) that can be rubbed on baby's skin after bath to retard moisture loss from baby's skin or to reduce harmful diaper rash caused by urine penetration. It is well known that water and oil cannot form a stable mixture after mixing. However, with the help of emulsifiers or small solid particles, water and oil can form stable systems called emulsions or Pickering emulsions. The traditional emulsions are surfactant emulsifier stabilized emulsions comprising water, at least one oil, and at least one surfactant emulsifier. The Pickering emulsions are solid particle stabilized emulsions comprising water, at least one oil, and at least one type of small solid particles. When there are emulsifiers or small particles present in the water-oil system, after mixing, the emulsifier molecules or the small solid particles can preferentially concentrate on the surfaces of the dispersed droplets, forming a protective layer against coagulation (fusion) of the dispersed droplets, thus the system is stabilized. Emulsions usually exist in two types, either oil-in-water (O/W) or water-in-oil (W/O). An O/W emulsion is a dispersion of fine oil droplets in water. A W/O emulsion is a dispersion of fine water droplets in oil. Typically other additives can be added to the emulsion system. Hydrophilic (i.e., water-loving) additives preferentially dissolve in water to form a water phase. Hydrophobic (water-hating) or lipophilic (oil-loving) additives preferentially dissolve in oil to form an oil phase.

Different applications have different stability requirements. In most applications, long term product stability, such as two years shelf life at room temperature (equivalently 4 weeks at 40° C. or 2 weeks at 50-54° C.), is required. It is known that it is more difficult to formulate a stable Pickering emulsion than a stable traditional emulsion. Therefore, the products based on Pickering emulsions are rare. In addition to surfactant emulsifiers and small solid particles, an acrylates/C10-30 alkyl acrylate crosspolymer (trade name Pemulen™ produced by Lubrizol) is sold as a primary oil-in-water emulsifier. The advantages of using a polymer instead of a surfactant emulsifier are that a polymer usually has lower toxicity and is more efficient. Acrylates/C10-30 alkyl acrylate crosspolymer is an effective aqueous thickener. Water thickened by this thickener can disperse oil and emulsify small oil droplets with adequate mixing. However, acrylates/C10-30 alkyl acrylate crosspolymer can only emulsify oil in water to prepare an O/W emulsion; it cannot be used to prepare a W/O emulsion.

There is a need for a thickener that is robust towards many organic liquids including plant derived oils and organic sunscreen actives in terms of thickening, that can thicken the organic liquids at ambient temperature, that can suspend solid or liquid particles without the need for dispersant or surfactant emulsifiers, wherein the thickening property is not affected at elevated temperature (40-50° C.), and that can be used in personal care formulations such as thickened oils, surfactant-free W/O and O/W emulsions, and solid particle suspensions in surfactant-free emulsions.

### SUMMARY OF THE INVENTION

We have discovered, unexpectedly, that a cross-linked (meth)acrylate copolymer can thicken various oils useful in personal care formulations.

We have also discovered, unexpectedly, that the thickened oils can suspend small aqueous droplets, forming stable surfactant-free W/O emulsions.

We have also discovered, unexpectedly, that such stable surfactant-free W/O emulsions are able to suspend solid particulates such as metal oxides, forming a suspension.

We have also discovered, unexpectedly, that the thickened oils can stabilize surfactant-free O/W emulsions formed with aqueous thickeners.

We have further discovered, unexpectedly, the oil thickeners of the present invention can enhance the SPF value in sunscreen formulations.

We have further discovered, unexpectedly, the oil thickeners of the present invention have water-proofing properties.

### DETAILED DESCRIPTION OF THE INVENTION

Throughout the present disclosure, the following words are used interchangeably: organic liquid, oil, and oil carrier; rheology modifier and thickener; and polymeric rheology modifier and polymer.

Throughout the present disclosure, the terms “cosmetically acceptable” and “dermatologically acceptable” mean compatible with skin tissue such as facial or bodily skin, and keratin materials such as the hair, the eyelashes, the eyebrows and the nails, and having a color, odor and feel and which does not generate unacceptable discomfort (stinging, tautness or redness) liable to dissuade the consumer from using the composition.

The Polymeric Rheology Modifiers.

The polymeric rheology modifiers selected for the formulations of the disclosure will be polymers that are dermatologically acceptable for use in personal care formulations.

For the polymeric rheology modifiers of the disclosure to be suitable for modifying the rheology of the oil component of personal care formulations, the polymer is preferably soluble in the oil carrier of the formulation. In the context of the present disclosure, a polymer is soluble in an oil if at least 3 wt % of polymer can be dissolved in the oil. Solubility can be determined by adding 3 wt % of a polymer to the oil and observing the clarity of the oil with the naked eye. The oil with dissolved polymer will be clear or have a slight translucent color or turbidity in it due to light scattering, but will not contain detectable polymer particles or a separate polymer-rich phase.

The polymeric rheology modifier is obtainable by co-polymerizing a monomer mixture comprising at least one alkyl (meth)acrylate and at least one of the following monomers:
a bicyclic (meth)acrylate ester different from the alkyl (meth)acrylate, and
an aromatic vinyl monomer.

In one embodiment, the polymeric rheology modifier is a cross-linked polymer comprising at least one alkyl (meth)acrylate monomer and at least one cyclic monomer, wherein the cyclic monomer is selected from the group consisting of a bicyclic (meth)acrylate ester, an aromatic vinyl monomer, and combinations thereof.

In one aspect, the cross-linked polymeric rheology modifier is obtainable by co-polymerizing at least two of the following ethylenically unsaturated monomers:
a bicyclic (meth)acrylate ester,
an alkyl (meth)acrylate, and
an aromatic vinyl monomer.

In one aspect, the polymeric rheology modifier is a cross-linked copolymer comprising two or more monomers selected from a bicyclic (meth)acrylate ester, a lower alkyl (meth)acrylate, a fatty alkyl (meth)acrylate, and an aromatic vinyl monomer, as long as the monomers comprising the copolymer include a bicyclic (meth)acrylate ester and/or an aromatic vinyl monomer.

For purposes of this disclosure, the term “lower alkyl (meth)acrylate” means C₁-C₆ alkyl (meth)acrylate.

For purposes of this disclosure, the term “fatty alkyl (meth)acrylate” means C₈-C₂₄ alkyl (meth)acrylate.

For purposes of this disclosure, the term “total alkyl (meth)acrylate” means the total of the weight percentages of any lower alkyl (meth)acrylates and fatty alkyl (meth)acrylates present in the copolymer.

In one aspect, the polymeric rheology modifier is a cross-linked copolymer comprising at least one cyclic monomer and at least one alkyl (meth)acrylate monomer, wherein
said at least one cyclic monomer is selected from the group consisting of a bicyclic (meth)acrylate ester, an aromatic vinyl monomer, and combinations thereof; and
said at least one alkyl (meth)acrylate monomer is selected from the group consisting of a lower alkyl (meth)acrylate, a fatty alkyl (meth)acrylate, and combinations thereof.

In one aspect, the polymeric rheology modifier comprises 5 to 50 wt % bicyclic (meth)acrylate ester, 25 to 70 wt % total alkyl (meth)acrylate, and 10 to 40 wt % aromatic vinyl monomer. In another embodiment, the rheology modifier comprises 20 to 70 wt % bicyclic (meth)acrylate ester, and 30 to 80 wt % total alkyl (meth)acrylate.

In an embodiment, the bicyclic (meth)acrylate ester is isobornyl methacrylate (IBOMA), the lower alkyl (meth)acrylate is isobutyl methacrylate (IBMA), and the aromatic vinyl monomer is styrene.

In another aspect, the rheology modifier is a cross-linked copolymer comprising a lower alkyl (meth)acrylate and/or a fatty alkyl (meth)acrylate and the rheology modifier is obtainable by co-polymerizing at least two of the following monomers:
a bicyclic (meth)acrylate ester,
a lower alkyl (meth)acrylate,
a fatty alkyl (meth)acrylate, and
an aromatic vinyl monomer,
   as long as the monomers comprising the copolymer include a bicyclic (meth)acrylate ester and/or an aromatic vinyl monomer.

In one aspect, the polymeric rheology modifier comprises 10 to 30 wt % bicyclic (meth)acrylate ester, 10 to 25 wt % lower alkyl (meth)acrylate, 30 to 40 wt % fatty-alkyl (meth)acrylate, and 15 to 30 wt % aromatic vinyl monomer. In an embodiment, the bicyclic (meth)acrylate ester is isobornyl methacrylate, the lower alkyl (meth)acrylate is isobutyl methacrylate, the fatty alkyl (meth)acrylate is lauryl methacrylate and the aromatic vinyl monomer is styrene.

In one embodiment, the cross-linked polymeric rheology modifier comprises isobornyl methacrylate and isobutyl methacrylate.

In one embodiment, the cross-linked polymeric rheology modifier comprises styrene and isobutyl methacrylate.

In one embodiment, the cross-linked polymeric rheology modifier comprises styrene, isobutyl methacrylate and lauryl methacrylate.

In one embodiment, the cross-linked polymeric rheology modifier comprises isobornyl methacrylate, styrene, and isobutyl methacrylate.

In one embodiment, the cross-linked polymeric rheology modifier comprises isobornyl methacrylate, isobutyl methacrylate and lauryl methacrylate.

In one embodiment, the cross-linked polymeric rheology modifier comprises isobornyl methacrylate, isobutyl methacrylate and ethylhexyl methacrylate.

In one embodiment, the cross-linked polymeric rheology modifier comprises isobornyl methacrylate, styrene, isobutyl methacrylate and lauryl methacrylate.

Throughout this document, the weight percentages of the monomer that constitute the copolymer are based on the total weight of the monomers used, whereby the total weight of the monomers adds up to 100 wt %.

The bicyclic (meth)acrylate ester of the disclosure contains a (meth)acryloyl radical bonded to a six-membered carbon atom bridged ring and said group of monomers include products like decahydronaphthyl (meth)acrylates, and adamantyl (meth)acrylates, but preferred are products according to formula (I).

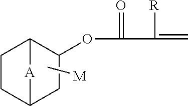

wherein

R is H or —CH₃,

A is —CH₂—, —CH(CH₃)— or —C(CH₃)₂—, and

one or more M is covalently bonded to any carbon of the bicyclic rings, preferably to a carbon atom of the six-membered ring, and is selected from the group consisting of hydrogen, halogen, methyl and methylamino group or a plurality thereof. Non-limiting examples of the bicyclic (meth)acrylate esters include isobornyl (meth)acrylate, bornyl (meth)acrylate, fenchyl (meth)acrylate, isofenchyl (meth)acrylate, norbornyl (meth)acrylate, cis, (endo) 3-methylamino-2-bornyl (meth)acrylate, 1,4,5,6,7,7-hexachlorobicyclo [2.2.1]-hept-5-ene-2-ol (meth)acrylate (HCBOMA) and 1,4,5,6,7,7-hexachlorobicyclo [2.2.1]-hept-5-ene-2 methanol (meth)acrylate (HCBMA), and mixtures of such bicyclic (meth)acrylates. Preferably the bicyclic (meth)acrylate ester of the disclosure is a bridged bicyclic (meth)acrylate ester. For purposes of this disclosure, a bridged bicyclic monomer means a monomer with two rings that share three or more atoms, separating the two bridgehead atoms by a bridge containing at least one atom. A suitable bicyclic (meth)acrylate ester is isobornyl methacrylate. The bicyclic (meth)acrylate esters are known per se and may be prepared in known fashion or may be obtained from commercial sources. The bicyclic (meth)acrylate is preferably chosen from monomers which, when polymerized, form a homopolymer that is soluble in the oil carrier of the sunscreen formulation.

Alkyl (meth)acrylates of the disclosure include lower alkyl (meth)acrylates, fatty-alkyl (meth)acrylates and mixtures thereof. In an embodiment, the alkyl (meth)acrylates are linear or branched. In one embodiment the alkyl (meth)acrylates are substituted.

Lower alkyl (meth)acrylates of the disclosure are C₁-C₆ alkyl (meth)acrylates. More particularly, lower alkyl (meth)acrylates of the disclosure are compounds wherein a (meth)acryloyl radical is bonded to a lower alkyl group, herein defined as a C₁-C₆ alkyl group, which can be linear or branched, substituted or unsubstituted, saturated or unsaturated. Lower alkyl (meth)acrylates of the disclosure include compounds such as methyl (meth)acrylate, ethyl (meth)acrylate, propyl (meth)acrylate, butyl (meth)acrylate, isobutyl (meth)acrylate, pentyl (meth)acrylate, isopentyl (meth)acrylate and hexyl (meth) acrylate. A preferred lower alkyl (meth)acrylate is isobutyl (meth)acrylate. The lower alkyl (meth)acrylate is preferably chosen from monomers which, when polymerized, form a homopolymer that is soluble in one or more of the oils of the formulations of the disclosure, and combinations thereof. When the homopolymer that is formed from the lower alkyl methacrylate is not soluble in the oils of the present formulations, the amount of this monomer in the polymeric rheology modifier is preferably limited to less than about 60%, more preferably less than 50% and more preferably less than about 40% by weight of the polymer.

Fatty-alkyl (meth)acrylates of the disclosure are C₈-C₂₄ alkyl (meth)acrylates. More particularly, the fatty-alkyl (meth)acrylates of the disclosure are compounds wherein a (meth)acryloyl radical is bonded to a fatty alkyl group, herein defined as a C₈-C₂₄ alkyl group, which can be linear or branched, substituted or unsubstituted, and saturated or unsaturated. Examples of a fatty alkyl (meth)acrylate include 2-ethylhexyl (meth)acrylate, decyl (meth)acrylate, isodecyl (meth)acrylate, lauryl (meth)acrylate, methacrylate ester 13.0 (CAS#: 90551-76-1), tetradecyl (meth)acrylate, hexadecyl (meth)acrylate, methacrylate ester 17.4 (CAS#: 90551-84-1), and stearyl (meth)acrylate. Preferred fatty-alkyl (meth)acrylates are chosen from monomers which, when polymerized, form a homopolymer which is soluble in the one or more of the oils of the formulations of the disclosure, and combinations thereof. In another embodiment 2-ethylhexyl (meth)acrylate, isodecyl (meth)acrylate, lauryl (meth)acrylate, methacrylate ester 13.0 (CAS#: 90551-76-1), methacrylate ester 17.4 (CAS#: 90551-84-1), and/or stearyl (meth)acrylate is used. Suitably lauryl methacrylate or 2-ethylhexyl (meth)acrylate is used.

The aromatic vinyl monomers of the disclosure contain a vinyl group bonded to an aromatic group. Examples include styrene, substituted styrene, vinyl naphthalene, and mixtures thereof. Preferred substituted styrenes include ortho-, meta- and/or para-alkyl, alkyloxy or halogen substituted styrenes, such as methyl styrene, 4-tert-butyl styrene, tert-butyloxy styrene, 2-chlorostyrene and 4-chlorostyrene. The preferred aromatic vinyl monomer is styrene. The use of styrene can increase the Tg of the polymer and reduce the cost. When the homopolymer that is formed from the aromatic vinyl monomer is not soluble in one or more of the oils of the formulations of the disclosure, and combinations thereof, the amount of this monomer in the polymeric rheology modifier is preferably limited to less than about 60%, more preferably less than 50% and more preferably less than about 40% by weight.

Other ethylenically unsaturated monomers different from the monomers above can also be included in the polymeric rheology modifier. These include but are not limited to monomers such as (meth)acrylic acid, maleic acid, 2-acrylamido-2-methylpropane, dimethylaminoethyl methacrylate, dimethylaminoethyl acrylate, N-[3-(dimethylamino) propyl] methacrylamide, N-[3-(dimethylamino) propyl] acrylamide, (3-acrylamidopropyl)-trimethyl-ammonium chloride, methacrylamido propyl trimethyl ammonium chloride, (meth)acrylamide, N-alkyl (meth)acrylamides, N-vinyl pyrrolidone, vinyl formamide, vinyl acetamide, and N-vinyl caprolactams. When one of these other monomers contains a hydroxyl, acid, basic nitrogen, or heterocylic functionality it is preferred that the polymer rheology modifier contain less than 2.5%, more preferably, less than 1.5% and most preferably less than 1% by weight of these monomers.

In another aspect, the polymeric rheology modifier of the disclosure is largely free of the polymerized residues of polar monomers. Polar monomers are defined as monomers that contain hydroxyl, carboxylic acid, nitrogen, or heterocyclic functionality.

In another aspect, the polymeric rheology modifiers are cross-linked polymers and further comprise suitable cross-linking monomers. Cross-linking monomers (or cross-linkers) contain two or more ethylenically unsaturated functionalities. These include, but are not limited to divinyl benzene, 1,6-hexanediol di(meth)acrylate, ethylene glycol di(meth)acrylate, propylene glycol di(meth)acrylate, trimethylol propane tri(meth)acrylate, trimethylolpropane triacrylatel, trimethylol propane diallyl ether, trimethylol propane triallyl ether, pentaerythritol triallyl ether, pentaerythritol tetraallyl ether, and pentaerythritol tri(meth)acrylate. The amount of cross-linker optionally present in the polymeric rheology modifier, based on the total weight of all monomers in the polymer, is from about 200 mg/kg to about 2000 mg/kg, preferably about 200 mg/kg to about 1500 mg/kg, more preferably about 300 mg/kg to about 1000 mg/kg, more preferably about 350 mg/kg to about 650 mg/kg. In some preferred embodiments, the amount of cross-linker in the polymeric rheology modifier is 200 mg/kg, or 220 mg/kg, or 240 mg/kg, or 260 mg/kg, or 280 mg/kg, or 300 mg/kg, or 320 mg/kg, or 340 mg/kg, or 360 mg/kg, or 380 mg/kg, 400 mg/kg, or 420 mg/kg, or 440 mg/kg, or 460 mg/kg, or 480 mg/kg, 500 mg/kg, or 520 mg/kg, or 540 mg/kg, or 560 mg/kg, or 580 mg/kg, 600 mg/kg, or 620 mg/kg, or 640 mg/kg, or 660 mg/kg, or 680 mg/kg, or 700 mg/kg, or 720 mg/kg, or 740 mg/kg, or 760 mg/kg, or 780 mg/kg, 800 mg/kg, or 820 mg/kg, or 840 mg/kg, or 860 mg/kg, or 880 mg/kg, 900 mg/kg, or 920 mg/kg, or 940 mg/kg, or 960 mg/kg, or 980 mg/kg, or 1000 mg/kg, or 1020 mg/kg, or 1040 mg/kg, or 1060 mg/kg, or 1080 mg/kg, or 1100 mg/kg, or 1120 mg/kg, or 1140 mg/kg, or 1160 mg/kg, or 1180 mg/kg, or 1200 mg/kg, or 1220 mg/kg, or 1240 mg/kg, or 1260 mg/kg, or 1280 mg/kg, or 1300 mg/kg, or 1320 mg/kg, or 1340 mg/kg, or 1360 mg/kg, or 1380 mg/kg, or 1400 mg/kg, or 1420 mg/kg, or 1440 mg/kg, or 1460 mg/kg, or 1480 mg/kg, or 1500 mg/kg, or 1520 mg/kg, or 1540 mg/kg, or 1560 mg/kg, or 1580 mg/kg, or 1600 mg/kg, or 1620 mg/kg, or 1640 mg/kg, or 1660 mg/kg, or 1680 mg/kg, or 1700 mg/kg, or 1720 mg/kg, or 1740 mg/kg, or 1760 mg/kg, or 1780 mg/kg, or 1800 mg/kg, or 1820 mg/kg, or 1840 mg/kg, or 1860 mg/kg, or 1880 mg/kg, or 1900 mg/kg, or 1920 mg/kg, or 1940 mg/kg, or 1960 mg/kg, or 1980 mg/kg, or 2000 mg/kg.

The amount of cross-linker will be selected to optimize the ability of the polymer to thicken the oil. For formulations that contain suspended solid or liquid particulates, the amount of cross-linker also will be selected to optimize the ability of the polymer to suspend the particulates. If the level of cross-linker is either too low or too high the particulates will not remain in a stable dispersion. The amount of cross-linker that will optimize system performance may vary depending on the selection of monomers, the relative proportions of the monomers in the polymer, the oil used as a carrier in the formulation, the size and type of particulates to be suspended, and other ingredients to be included in the formulation.

Where the polymeric rheology modifiers are crosslinked, it will be understood that the wt % values of the monomers as stated herein are approximate so as to allow for the presence of the monomeric crosslinker.

Preferably, the glass transition temperatures (Tg) of the polymeric rheology modifier is high enough that the polymer can be isolated and handled as a solid at room temperature (approximately 22° C.). Preferably the Tg of the polymeric rheology modifier is greater than about 45° C., more preferably greater than about 60° C. and more preferably greater than about 75° C. Tg can be measured using standard procedures such as differential scanning calorimetry. For the Tg values described herein, the Tg of the polymer was determined by placing a vial containing the polymer powder to be measured into a hot water bath (e.g., 75° C.) for 10 minutes. If the powder remained free flowing after 10 minutes in the hot water bath, the Tg of the powder was determined to be at least the temperature of the water bath. The temperature of the water bath was increased incrementally until the polymer was no longer free flowing to determine the Tg where appropriate. In other instances, a Tg was determined to be “greater than” the last water bath temperature in cases were an upper end transition temperature was not determined. The polymeric rheology modifiers of the present disclosure typically have a Tg>75° C.

The weight average molecular weight (Mw) of the copolymer of the invention, when measured in accordance with the method described below in Example 16 is preferably at least 20,000,000 Dalton (D), suitably at least 50,000,000 (D); 100,000,000 (D); 150,000,000; and/or at least 200,000,000 D.

The polymeric rheology modifier of the disclosure may be synthesized by conventional methods for vinyl addition polymerization known to those skilled in the art, such as, but not limited to, solution polymerization, precipitation polymerization, and dispersion polymerizations, including suspension polymerization and emulsion polymerization. The preferred process is emulsion polymerization.

In an embodiment in which the polymeric rheology modifiers of the disclosure are formed by emulsion polymerization, one or more monomers are dispersed, in one step or multiple steps, in an aqueous phase and polymerization is initiated using a water soluble initiator. The monomers are typically water insoluble or very poorly soluble in water, and a surfactant or soap is used to stabilize the monomer droplets in the aqueous phase. Polymerization occurs in the swollen micelles and latex particles. Other ingredients that might be present in an emulsion polymerization include chain transfer agents such as mercaptans (e.g. dodecyl mercaptan) to control molecular weight, small amounts of water soluble organic substances such as but not limited to acetone, cyclodextrin, glycols, 2-butanone, methanol, ethanol, and isopropanol, to adjust the polarity of the aqueous phase, and electrolytes to control pH. Suitable initiators include alkali metal or ammonium salts of persulfate such as ammonium persulfate, water-soluble azo compounds such as 2,2′-azobis(2-aminopropane)dihydrochloride, and redox systems such as Fe(II) and cumene hydroperoxide, and tert-butyl hydroperoxide-Fe(II)-sodium ascorbate. Suitable surfactants include anionic surfactants such as fatty acid soaps (e.g. sodium or potassium stearate), sulfates and sulfonates (e.g. sodium dodecyl benzene sulfonate or calcium dodecylbenzene sulfonate), sulfosuccinates (e.g. dioctyl sodium sulfosuccinate); non-ionic surfactants such as octylphenol ethoxylates and linear and branched alcohol ethoxylates, and alkylamine alkoxylates; cationic surfactants such as cetyl trimethyl ammonium chloride; and amphoteric surfactants. Anionic surfactants and combinations of anionic surfactants and non-ionic surfactants are most commonly used. Polymeric stabilizers such as poly(vinyl alcohol-co-vinyl acetate) can also be used as surfactants. The solid polymer product free of the aqueous medium can be obtained by a number of processes including destabilization/coagulation of the final emulsion followed by filtration, solvent precipitation of the polymer from latex, or spray drying of the latex.

If the powder particle size is too large (e.g., more than mesh size 60 or 250 microns), the powder particles require a long time to dissolve in the oil carrier of the sunscreen formulation. In some cases, if the sample is left un-agitated, the swollen polymer particles could stick together, preventing further dissolution. Hence, the particle size of the powder is preferably smaller than 60 mesh size, more preferably smaller than 100 mesh size (or ˜150 microns). The polymeric rheology modifiers of the present disclosure are preferably a free flowing powder obtained by a spray drying process or by any suitable drying processes known in the art. However, a liquid latex of polymeric rheology modifier can also be used if the application can tolerate the presence of some water.

As used herein, mesh size refers to standard United States (US) mesh size. The mesh size number indicates the number of openings located along 1 linear inch of mesh.

In one aspect, the polymeric rheology modifiers used in the personal care formulations of the present disclosure comprise at least 5 wt % of bicyclic (meth)acrylate ester, in another aspect at least 10 wt %, in another aspect at least 20 wt %, in another aspect at least 40 wt %, in still another aspect at least 60 wt %, and in still another aspect at least 70 wt %. In one embodiment the preferred range of bicyclic (meth)acrylate ester present in the rheology modifier is 5 to 50 wt %. In another embodiment, the preferred range of bicyclic (meth)acrylate ester present in the rheology modifier is 10 to 30 wt %. In another embodiment, the preferred range of bicyclic (meth)acrylate ester present in the rheology modifier is 20 to 70 wt %, or 25 to 60 wt %, or 30 to 55 wt %. In some preferred embodiments, the amount of bicyclic (meth)acrylate ester in the polymeric rheology modifier is 5 wt %, or 10 wt %, or 15 wt % or 20 wt %, or 25 wt %, or 30 wt %, or 35 wt % or 40 wt %, or 45 wt %, or 50 wt %, or 55 wt % or 60 wt %, or 65 wt %, or 70 wt %, or 75 wt %.

In another aspect, the polymeric rheology modifiers used in the personal care formulations of the present disclosure comprise at least 25 wt % of total alkyl (meth)acrylates, in another embodiment at least 35 wt %, in another embodiment at least 50 wt %, in another embodiment at least 65 wt %, and in another embodiment at least 80 wt %. In one embodiment the preferred range of total alkyl (meth)acrylate present in the rheology modifier is 25 to 70 wt %. In another embodiment, the preferred range of total alkyl (meth)acrylate present in the rheology modifier is 30 to 80 wt %, or 40 to 75 wt %, or 45 to 70 wt %. In some preferred embodiments, the amount of total alkyl (meth)acrylate in the polymeric rheology modifier is 25 wt %, or 30 wt %, or 35 wt % or 40 wt %, or 45 wt %, or 50 wt %, or 55 wt % or 60 wt %, or 65 wt %, or 70 wt %, or 75 wt % or 80 wt %, or 85 wt %, or 90 wt %.

In another aspect, polymeric rheology modifiers used in the personal care formulations of the present disclosure comprise at least 10 wt % of lower alkyl (meth)acrylates, in another embodiment at least 15 wt %, in another embodiment at least 20 wt %, in another embodiment at least 25 wt %, or at least 30 wt %, or at least 40 wt %, or at least 50 wt %, or at least 60 wt %, or at least 65 wt %, or at least 70 wt %, or at least 75 wt %, or at least 80 wt %, or at least 85 wt %, or at least 90 wt %. In one embodiment the preferred range of lower alkyl (meth)acrylate present in the rheology modifier is 50 to 90 wt %.

In another aspect, the polymeric rheology modifiers used in the personal care formulations of the present disclosure comprise at least 10 wt % of fatty alkyl (meth)acrylates, or at least 20 wt %, or at least 30 wt %, or at least 35 wt %, or at least 40 wt %. In an embodiment the preferred range of fatty alkyl (meth)acrylate present in the rheology modifier is 20 to 40 wt %.

In another aspect, the polymeric rheology modifiers used in the personal care formulations of the present disclosure comprise less than about 40 wt % of aromatic vinyl monomers, in another embodiment less than about 35 wt %, less than about 30 wt %, less than about 25 wt %, in another embodiment less than about 20 wt %, and in another embodiment less than about 15 wt %. In another embodiment, the preferred range of aromatic vinyl monomer present in the rheology modifier is 10 to 40 wt %, or 15 to 30 wt %.

In an aspect, the polymeric rheology modifier used in the personal care formulations of the present disclosure is polymerized from a reaction mixture comprising:
about 5 to about 50 wt % of the bicyclic (meth)acrylate ester
about 25 to 85 wt % of the lower alkyl (meth)acrylate, and
10 to 40 wt % of the aromatic vinyl monomer.

In another aspect, the polymeric rheology modifier used in the personal care formulations of the present disclosure is polymerized from a reaction mixture comprising:
20 to 70 wt %, preferably 25 to 60 wt %, and more preferably 30 to 55 wt % bicyclic (meth)acrylate ester, and
30 to 80 wt %, preferably 40 to 75 wt %, and more preferably 45 to 70 wt % total alkyl (meth)acrylate.

In another aspect, the polymeric rheology modifier used in the personal care formulations of the present disclosure is polymerized from a reaction mixture comprising:
10 to 30 wt % bicyclic (meth)acrylate ester,
10 to 25 wt % lower alkyl (meth)acrylate,
30 to 40 wt % fatty-alkyl (meth)acrylates, and
15 to 30 wt % aromatic vinyl monomer.

In another aspect, the polymeric rheology modifier used in the personal care formulations of the present disclosure is obtainable by copolymerizing a lower alkyl (meth)acrylate monomer with at least one additional monomer selected from:
a bridged bicyclic (meth)acrylate ester monomer,
an aromatic vinyl monomer, and
a fatty-alkyl (meth)acrylate monomer,
   as long as the monomers comprising the copolymer include a bridged bicyclic (meth)acrylate ester and/or an aromatic vinyl monomer, and wherein each of said monomers can be substituted or unsubstituted.

Throughout this document, the weight percentages of the monomer that constitute the copolymer are based on the total weight of the monomers used, whereby the total weight of the monomers adds up to 100 wt %, except for the presence of cross-linker.

In the copolymer rheology modifiers of the disclosure, the monomers may be arranged in any fashion, such as in blocks or randomly. Preferably, the copolymer is a randomly arranged copolymer.

The rheology modifiers used in the personal care formulations of the present disclosure are preferably a free flowing powder obtained by a spray drying process or by any suitable drying processes known in the art. However, a liquid form can also be used.

Oils

The oils are selected from one or more oils that are dermatologically acceptable and suitable for use in a personal care formulation, and are preferably in the liquid state at the temperature of application. Non-limiting examples of oils suitable for use in the personal care formulations of the disclosure include triglycerides, esters, silicone oils with aromatic groups, organic sunscreen active ingredients, and aromatic compounds. Preferred triglycerides are plant derived oils including soybean oil, sunflower oil, safflower oil, canola oil, rapeseed oil, corn oil, macadamia oil, and tea oil. Non-limiting examples of triglycerides are caprylic capryl triglyceride, glycerol tri-2-ethylhexyl, and oils of various plants. More preferred triglycerides are caprylic capryl triglyceride and oils from various plants. More preferred oils from various plants are soy bean oil, rapeseed oil, canola oil, corn oil, and linseed oil.

Preferred esters are fatty acid esters, lactates, maleates, adipates, citrates, succinates, and benzoate esters, and salicylic acid esters. The fatty acid esters suitable for use in the formulations of the present disclosure refer to any dermatologically acceptable ester of an oil derived from vegetables or animals, including but not limited to hydrogenated and non-hydrogenated, epoxidized and non-epoxidized, soy methyl esters (SME), rapeseed methyl esters, canola methyl esters, methyl cocoate, safflower methyl esters, ricinoleic acid methyl esters, castor methyl esters, isopropyl myristate (ISPM), isopropyl palmitate, methyl oleate, and C₈-C₁₀ methyl esters. The fatty acid esters of the disclosure are preferably plant derived. The preferred lactates are 2-ethylhexyl lactate, butyl lactate and propyl lactate. The preferred maleate is diethyl maleate. The preferred adipate is dimethyl adipate. The preferred citrates are acetyl tributyl citrate, butyryltri-n-hexyl citrate, and tributyl citrate. The preferred succinate is diethyl succinate. The preferred benzoates are methyl benzoate, ethyl benzoate, and C12/15 benzoate. The preferred salicylic acid ester is butyloctyl salicylate.

The term “oil” can also include certain organic sunscreen active agents. For purposes of the present application, a “sunscreen active agent” or “sunscreen active” shall include all of those materials, singly or in combination, that are regarded as acceptable for use as active sunscreen ingredients based on their ability to absorb UV radiation. Such compounds are generally described as being UV-A, UV-B, or UV-A/UV-B active agents. Approval by a regulatory agency is generally required for inclusion of active agents in formulations intended for human use. Those active agents which have been or are currently approved for sunscreen use in the United States include organic and inorganic substances including, without limitation, para aminobenzoic acid, avobenzone, cinoxate, dioxybenzone, homosalate, menthyl anthranilate, octyl salicylate, oxybenzone, padimate O, phenylbenzimidazole sulfonic acid, sulisobenzone, trolamine salicylate, diethanolamine methoxycinnamate, digalloy trioleate, ethyl dihydroxypropyl PABA, glyceryl aminobenzoate, lawsone with dihydroxyacetone, red petrolatum. Examples of additional sunscreen actives that have not yet been approved in the US but are allowed in formulations sold outside of the US include ethylhexyl triazone, dioctyl butamido triazone, benzylidene malonate polysiloxane, terephthalylidene dicamphor sulfonic acid, disodium phenyl dibenzimidazole tetrasulfonate, diethylamino hydroxybenzoyl hexyl benzoate, bis diethylamino hydroxybenzoyl benzoate, bis benzoxazoylphenyl ethylhexylimino triazine, drometrizole trisiloxane, methylene bis-benzotriazolyl tetramethylbutylphenol, and bis-ethylhexyloxyphenol methoxyphenyltriazine, 4-methylbenzylidenecamphor, and isopentyl 4-methoxycinnamate. The preferred organic sunscreen active ingredients are octyl salicylate, ethylhexyl methoxycinnamate, homosalate, octocrylene, and menthyl anthranilate (meradimate). However, as the list of approved sunscreens is currently expanding, those of ordinary skill in the art will recognize that the invention is not limited to sunscreen active agents currently approved for human use but is readily applicable to those that may be allowed in the future.

Preferred organic sunscreen active ingredients are octyl salicylate, ethylhexyl methoxycinnamate, homosalate, octocrylene, and menthyl anthranilate (meradimate). Due to regulations in various countries, the amount of total organic sunscreen active ingredients in a formulation may be limited to less than 39%. Because of this limitation, if organic sunscreen active ingredients are used in the sunscreen formulations of the disclosure, preferably at least one other oil of the disclosure is also used in the formulation.

Preferred silicone oils with aromatic groups include phenyl trimethicone (Dow Corning 556 oil).

Mixtures of oils also can be used. Other dermatologically acceptable oils will be known to those skilled in the dermatological arts.

Other Ingredients

The personal care formulations of the disclosure can optionally contain additional cosmetically acceptable ingredients, as long as those ingredients are either soluble in the formulation or are in the form of particulates that exist in stable dispersions in the formulations.

Other dermatologically acceptable rheology modifiers that are soluble in oil can be used in the compositions of the present disclosure, and can be polymeric or non-polymeric. Non-limiting examples include the thickeners disclosed in “Diverse Technologies for Polymeric Oil Thickeners” posted on Mar. 14, 2014 by George Deckner at http://knowledge.ulprospector.com/388/pcc-diverse-technologies-polymeric-oil-thickeners, the contents of which are incorporated herein by reference in their entirety. Preferably they are selected from the following group of rheology modifiers: Asensa CL 300 (ethylene/vinyl acetate copolymer) and SC 401 (ethylene/acrylic acid copolymer) (both from Honeywell); Syncrowax ORM (Sorbitol/Sebacic Acid Copolymer Behanate) (Croda); Hostacerin DP (Dextrin Palmitate) (Clariant); Rheopearl ISK2/ISL2 (Stearoyl Inulin) (Chiba Flour Milling Ltd); Versagels (thickened oil gels) (Calumet Penreco); Intelimer IPA 13-6 and Intelimer IPA 13-1 (poly C10-30 alkyl acrylates) (Air Products and Chemicals); Kraton G1702 Polymer (styrene and ethylene/propylene diblock copolymer); hydrogenated castor oil; ethyl cellulose; Atlox Rheostrux 200; Atlox Rheostrux 100; Oleocraft LP-20 (Polyamide-8 from Croda).

The personal care formulations of the present invention may contain a wide range of additional, optional components which are referred to herein as “cosmetic components”, but which can also include components generally known as pharmaceutically active agents. The CTFA Cosmetic Ingredient Handbook, Seventh Edition, 1997 and the Eighth Edition, 2000, which is incorporated by reference herein in its entirety, describes a wide variety of cosmetic and pharmaceutical ingredients commonly used in skin care compositions, which are suitable for use in the compositions of the present invention. Examples of these functional classes disclosed in this reference include: abrasives, antioxidants, vitamins, biological additives, chemical additives, colorants, cosmetic astringents, cosmetic biocides, drug astringents, external analgesics, film formers, fragrance components, sunscreen agents, ultraviolet light absorbers, and SPF boosters.

Fragrances are aromatic substances which can impart an aesthetically pleasing aroma to the sunscreen composition. Typical fragrances include aromatic materials extracted from botanical sources (i.e., rose petals, gardenia blossoms, jasmine flowers, etc.) which can be used alone or in any combination to create essential oils. Alternatively, alcoholic extracts may be prepared for compounding fragrances. However, due to the relatively high costs of obtaining fragrances from natural substances, the modern trend is to use synthetically prepared fragrances, particularly in high-volume products. One or more fragrances can optionally be included in the sunscreen composition in an amount ranging from about 0.001 to about 5 weight percent, preferably about 0.01 to about 0.5 percent by weight.

Essential oils can be included in the compositions of the disclosure. The preferred essential oils are sandalwood oil, cedarwood oil, chamomile oil, vanilla oil, tea tree oil, eucalyptus oil, peppermint oil, bergamot oil, lavender oil, rosemary oil, rose oil, cinnamon oil, frankincense oil, lemongrass oil, geranium oil, orange oil, vetiver oil, lemon oil, jasmine oil, cedar oil, and grapefruit oil. Pine oil and d-limonene also can be used.

Advantageously, such essential oils also can be thickened by the cross-linked polymeric rheology modifiers of the disclosure.

Additional preservatives may also be used if desired and include well known preservative compositions such as benzyl alcohol, phenyl ethyl alcohol and benzoic acid, diazolydinyl, urea, chlorphenesin, iodopropynyl and butyl carbamate, among others.

The personal care formulations of the invention may further include insect repelling components. The most widely used insect repelling active agent for personal care products is N,N-Diethyl-m-toluamide, frequently called “DEET” and available in the form of a concentrate containing at least about 95 percent DEET. Other synthetic chemical repellents include ethyl butylacetylaminoproprionate (also known as IR 3535), dimethyl phthalate, ethyl hexanediol, indalone, di-npropylisocinchoronate, bicycloheptene, dicarboximide and tetrahydrofuraldehyde. Certain plant derived materials also have insect repellent activity, including citronella oil and other sources of citronella (including lemon grass oil), limonene, rosemary oil and eucalyptus oil. Choice of an insect repellent for incorporation into the emulsions will frequently be influenced by the odor of the repellent. The amount of repellent agent used will depend upon the choice of agent; DEET is useful at high concentrations, such as up to about 15 percent or more, while some of the plant-derived substances are typically used in much lower amounts, such as 0.1 percent or less.

In one aspect of the disclosure, personal care formulations can be in the form of oil-in-water emulsions, or water-in-oil emulsions. Advantageously, the emulsions can be free of other surfactants. In one embodiment, the emulsions can be free of sulfate-based surfactants, including without limitation sulfate-based surfactants such as sodium lauryl sulfate or sodium laureth sulfate. The polymeric rheology modifiers as disclosed herein are soluble in the oil phase of the emulsion. Without being bound by theory, it is believed that the solubililzed rheology modifier stabilizes the emulsion, whether it be a water-in-oil emulsion or an oil-in-water emulsion. The emulsions may also include particles suspended therein, such as metal oxide sunscreen particles, or other suspended particles as are known in the personal care arts.

Various methods of preparing personal care formulations are well-known in the art. One exemplary method of making the personal care formulations of this application comprises the step of mixing the polymeric rheology modifier in the oil carrier with adequate agitation and for a sufficient period of time (usually <30 minutes) to dissolve the rheology modifier in the oil carrier. If particulates are used, the particulates are then mixed into the formulation with adequate agitation and for a sufficient period of time. Additional ingredients can be added to the formulation either before the rheology modifier is added to the oil carrier, or after the rheology modifier has been added but before the particulates are added, or after the particulates are added. Another exemplary method of preparing a personal care formulation (i.e., a W/O emulsion) comprises the steps of mixing the oil and water at vigorous shearing first, followed by adding the rheology modifier with reduced shearing and mixing the system until all rheology modifier are dissolved. In most cases, the formulations can be prepared at room temperatures using an overhead stirrer or by shaking with hands or machines. In few cases, if the rheology modifier takes a long time (>2 hours) to dissolve, heating may be used to facilitate dissolution of the rheology modifier into the oils. If homogenization is used, add the rheology modifier after homogenization or limit the homogenization duration to as short as necessary if the rheology modifier is in the formulation.

One particular application of the personal care formulations described herein to skin of a human will provide enhanced protection against deleterious effects of ultraviolet radiation (UVR). Thus, the subject invention further provides a method for protecting human skin against the deleterious effects of solar radiation, more particularly UVR, which method comprises topically applying thereto an effective amount of the compositions as described herein. An aesthetically beneficial result of exposure of skin to UVR (i.e., light radiation wavelengths of from 280 nm to 400 nm) is the promotion of tanning of the human epidermis. Another benefit of sun exposure comes from production of vitamin D within the skin. UVR is typically divided into UV-A (light wavelengths from 320 to 400 nm) and UV-B (wavelengths ranging from 280 to 320 nm) regions.

Overexposure to UV-B irradiation is generally understood to lead to skin burns and erythema. In addition, overexposure to UV-A radiation may cause a loss of elasticity of the skin and the appearance of wrinkles, promoting premature skin aging. Such irradiation promotes triggering of the erythemal reaction or amplifies this reaction in certain individuals and may even be the source of phototoxic or photoallergic reactions. It is increasingly believed that overexposure to UV-A may also lead to melanoma. Thus, the application of the compositions of the invention to the skin of an individual will provide enhanced UVR photoprotection (UV-A and/or UV-B) of the skin of the individual.

Rheology Modification

The rheology modifiers of the disclosure have the ability to thicken personal care oils for use in either oil-based formulations or emulsion-based formulations. For the purposes of this disclosure, thickening a composition means to increase the viscosity of the oil by at least five times, or at least 10 times, preferably at least 20 times, and more preferably at least 50 times in the presence of 3% w/w or less of the rheology modifier compared to the same oil in the absence of the rheology modifier. Viscosity is measured by Brookfield viscometers at 10 rpm at 22° C., using either a Brookfield DV-II+Viscometer or a Brookfield DV-I Prime Viscometer as indicated in the examples below. Typically, the viscosity of an oil is low and pure oils are generally Newtonian fluids. For example, fatty acid ester is less than 10 mPas and soy oil is ˜50 mPas and they are Newtonian.

The compositions of the disclosure can be a clear liquid, a lotion, or a cream, and preferably are stable, exhibiting no separation for at least 2 weeks at 50-54° C.

The viscosity requirement of typical personal care formulations depends on the preference of each manufacturer and it can range from very low (e.g., less than 1000 cps) to very high (e.g., non-flowable cream or gel).

In one aspect, the formulations are thickened organic liquids comprising:
A) 0.1-30%, preferably 1-20%, more preferably 2-10% cross-linked (meth)acrylate copolymer of the disclosure; and
B) 70-99.9%, preferably 80-99%, more preferably 90-98% organic liquids

In one aspect, the formulations are emulsions comprising:
C) 5-90%, preferably 10-70%, more preferably 20-50% water;
D) 5-90%, preferably 20-80%, more preferably 40-70% oil; and
E) greater than 0.2%, preferably greater than 1%, and more preferably greater than 3% cross-linked (meth)acrylate copolymer;
F) And optionally 0-1%, preferably, 0-0.5% aqueous thickeners such as acrylates/C10-30 alkyl acrylate crosspolymer.

In one aspect, the formulations are emulsions with particles suspended therein, the formulations comprising:
A) 5-90%, preferably 10-70%, more preferably 20-50% water;
B) 5-90%, preferably 20-80%, more preferably 40-70% oil; and
C) greater than 1%, preferably greater than 3%, and more preferably greater than 5% cross-linked (meth)acrylate copolymer; and
D) 2-20%, preferably, 5-15%, more preferably, 8-12% solid particulates;
E) And optionally 0-1%, preferably, 0-0.5% aqueous thickeners such as acrylates/C10-30 alkyl acrylate crosspolymer.

The personal care formulations comprising the cross-linked (meth)acrylate copolymer as disclosed herein can be any personal care formulation that can include a thickened oil, or a water-in-oil emulsion, or an oil-in-water emulsion with a thickened water. The formulations optionally can include suspended solid or liquid particulates, depending on the intended use of the formulation. Such formulations can include without limitation hair care products, such as shampoos, conditioners, and treatments; skin care products and sun care products; deodorants and antiperspirants; and color cosmetics. The personal care formulations herein can be in the form of oils, lotions, creams, gels, sticks, sprays, or solids. The personal care formulations herein are generally suitable for topical application to the skin and/or hair and thus generally comprises a physiologically acceptable medium, that is to say compatible with skin tissue such as facial or bodily skin, and keratin materials such as the hair, the eyelashes, the eyebrows and the nails. The personal care formulations will be cosmetically acceptable, that is to say having a color, odor and feel and which does not generate unacceptable discomfort (tingling, tightness, redness) liable to dissuade the consumer from using this formulation.

### EXAMPLES

### Example 1

### Synthesis of Cross-Linked Isobornyl Methacrylate—Isobutyl Methacrylate Polymeric Rheology Modifier

### Example 1A

Exemplary polymeric rheology modifiers were made using different combinations of monomers. Isobornyl methacrylate (IBOMA), isobutyl methacrylate (IBMA), 2-ethylhexylmethacrylate (2-EHMA) and isodecyl methacrylate (IsoC10 MA) were obtained from Sigma-Aldrich. Lauryl methacrylate (LMA) was methacrylic ester 13.0 obtained from Evonik (VISIOMER® terra C13-MA). All monomers are available from Evonik as well, including isobutyl methacrylate (VISIOMER® i-BMA), isobornyl methacrylate (VISIOMER® Terra IBOMA) 2-EHMA (VISIOMER® EHMA), and isodecyl methacrylate (VISIOMER® IDMA).

Exemplary polymeric rheology modifier (Synthesis Example 1) was prepared according to the following basic procedure.

TABLE 1A
Synthesis of Example 1A Polymeric Rheology Modifier

| Initial Charge: | WT | | wt % |
|---|---|---|---|
| Deionized water | 670.15 | g | 56.72 |
| Aerosol ® OT-75 PG (sodium dioctyl | 12.1 | g | 1.02 |
| sulfosuccinate, 75% in propylene glycol | | | |
| and water; available from Cytec) | | | |
| Co-solvent: | | | |
| Acetone | 168.79 | g | 14.28 |
| Monomer mix: | | | |
| Isobornyl methacrylate | 162.59 | g | 13.76 |
| Isobutyl methacrylate | 162.50 | g | 13.75 |
| 1,6-hexanediol diacrylate | 0.1625 | g | 0.01 |
| Oxidant solution: | | | |
| t-Butyl hydroperoxide, 0.0348 g/mL | 0.85 mL; 0.02958 g, | | 0.07 |
| solution in deionized water | active basis | | |
| Reductant solution: | | | |
| Deionized water | 3.70 | g | 0.31 |
| Sodium ascorbate | 0.0801 | g | 0.01 |
| Iron (II) sulfate heptahydrate, | 0.67 | g | 0.06 |
| 0.25% in deionized water | | | |
| Total | 1181.59 | g | 100 |

Polymerization Procedure

A 2 L, 4-neck round bottom flask was equipped with an overhead mechanical stirrer; a Y-tube equipped with a nitrogen purge outlet-topped condenser and a thermometer; and two septa. To the flask were charged deionized water and Aerosol OT-75 PG. Using a thermostat controlled water bath, the reaction temperature was brought to about 48° C. A 12 minute sub-surface nitrogen purge was then initiated via a needle inserted through one of the septa while maintaining a 200 rpm agitation rate.

While maintaining the nitrogen purge, the monomer mixture and acetone were charged to the reaction vessel. The sub-surface nitrogen purge was continued after the monomer/acetone addition.

In a separate container, a reductant solution consisting of sodium ascorbate and iron (II) sulfate heptahydrate dissolved in deionized water was prepared. The iron (II) sulfate heptahydrate was added after the ascorbate had dissolved and just before use of the reductant solution.

The reaction was purged for an additional 12 minutes after the monomer/acetone addition, and then the resulting dark blue ascorbate solution was added via syringe to the reaction vessel in one shot while maintaining the sub-surface nitrogen purge.

About 10 minutes after the addition of the reductant, 0.85 mL of a t-butyl hydroperoxide solution in water (0.0348 g/mL) was added to the reaction via syringe in one shot while maintaining the sub-surface nitrogen purge.

Within about 7 minutes, the onset of an exotherm was noted, and the sub-surface nitrogen purging was stopped in favor of above-surface nitrogen purging. As the reaction progressed, a bluish tint was noted in the emulsion, and it became increasingly more translucent, and a slight increase in viscosity was noted. The reaction temperature reached a maximum of about 56° C. (initial temp: 48° C.) before it began to subside after about 40 min. The reaction temperature was maintained thereafter at 48-50° C. using the water bath. After a total of 5 h reaction time, the reaction was cooled and poured through cheesecloth into a container.

The product was a milky liquid with a solid content of ˜29.0% (measured gravimetrically).

Solid polymer was isolated by adding the undiluted emulsion polymer to an excess of 0.5 N ammonium acetate in deionized water. The resulting precipitate was collected by vacuum filtration and washed extensively with water, and the solid was dried to a constant weight in a forced air oven at 60° C. Alternatively, solid polymer can be obtained by pan-drying or spray drying the liquid product at elevated temperatures with various techniques known to the skilled in the art. The solid obtained after drying can be further ground into a fine powder with various techniques known to the skilled in the art. The powder was passed through a 100 mesh screen in this example.

### Example 1B

### Semibatch Emulsion Polymerization Process

An additional exemplary polymeric rheology modifier was prepared using a semi-batch emulsion polymerization process.

TABLE 1B
Synthesis of Example 1B Polymeric Rheology Modifier

| | Wt. (g) | Wt. % |
|---|---|---|
| Monomer Pre-Emulsion: | | |
| Aerosol OT-75 PG | 4.32 | 0.88% |
| Deionized Water | 96.00 | 19.65% |
| Acetone | 8.00 | 1.64% |
| isobutyl methacrylate | 103.92 | 21.28% |
| isobornyl methacrylate | 56.00 | 11.46% |
| 1,6-hexanediol diacrylate | 0.0800 | 0.02% |
| Initial Charge: | | |
| Deionized water: | 80.00 | 16.38% |
| Acetone | 26.56 | 5.44% |
| Reductant Shot Solution: | | |
| sodium ascorbate | 0.07 | 0.01% |
| iron (II) sulfate heptahydrate, | 0.48 | 0.10% |
| 0.25% in deionized water | | |
| deionized water | 8.00 | 1.64% |
| Oxidant Shot Solution: | | |
| tert-butyl hydroperoxide, 70% | 0.02 | 0.00% |
| deionized water | 8.00 | 1.64% |
| Reductant Feed: | | |
| sodium ascorbate | 0.03 | 0.01% |
| iron (II) sulfate heptahydrate, | 0.16 | 0.03% |
| 0.25% in deionized water | | |
| deionized water | 33.90 | 6.94% |
| acetone | 14.40 | 2.95% |
| Oxidant Feed: | | |
| tert-butyl hydroperoxide, 70% | 0.02 | 0.00% |
| deionized water | 34.08 | 6.98% |
| acetone | 14.40 | 2.95% |
| total: | 488.45 | 100.00% |

Polymerization Procedure

A 2 L, 4-neck round bottom flask was equipped with an overhead mechanical stirrer; a Y-tube equipped with a nitrogen purge outlet-topped condenser and a thermometer; a 500 mL pressure equalized addition funnel, and a septum. To the flask were charged 80 g deionized water and 26.56 g acetone (Initial Charge).

In a separate beaker, the Monomer Pre-Emulsion was prepared. To a stirred solution of 4.32 g Aerosol OT-75 PG in 96.00 g deionized water and 8.00 g acetone was added a pre-made mixture of isobutyl methacrylate, isobornyl methacrylate, and 1,6-hexanediol diacrylate over a period of about 5 minutes with vigorous mechanical stirring (500-700 rpm); subsurface nitrogen purging was maintained throughout the addition. The addition was slow at first and then more rapid. Stirring was continued for an additional 5 minutes after the addition was complete; the sub-surface nitrogen purge was continued as well.

To the reaction flask was added 20% by weight of the Monomer Pre-Emulsion. The remaining 80% of the Monomer Pre-Emulsion was charged to the addition funnel, which was then capped with a stopper.

Using a thermostat controlled water bath, the reaction temperature was brought to about 48° C., and a sub-surface nitrogen purge was initiated via a needle inserted through one of the septa. The stirring speed was maintained at about 500 rpm for 15 minutes and then the stirring speed was reduced to about 260 rpm and held at this rate for the duration of the reaction.

When the reaction temperature had stabilized, the Reductant Shot Solution was added via syringe through the septum. After 10 minutes, the Oxidant Shot Solution was added via syringe through the septum. Within about 2 minutes of the addition of the Oxidant Shot Solution, an exotherm was noted and a blue tint was observed. About 5 minutes after the addition of the Oxidant Shot Solution, uniform continuous addition of the balance of the Monomer Pre-Emulsion over 2 h. was initiated. Simultaneously, the uniform and continuous addition of both the Reductant and Oxidant Feeds via syringe pumps over a period of 3 h was initiated.

The reaction temperature was maintained at 48-50° C. throughout the addition of the monomer pre-emulsion. The temperature was then raised to about 53° C. and then held at this temperature throughout the rest of the Reductant and Oxidant feed additions using the water bath and then for an additional 1 h after the Reductant and Oxidant Feeds had finished.

The product was a milky liquid with a solid content of 33.2% (measured gravimetrically).

Solid polymer was obtained by pan-drying at 60° C. in a forced air oven. Alternatively, solid product could be obtained by coagulation with salt (as in Synthesis Example 1A) or spray drying the liquid product at elevated temperatures with various techniques known to the skilled in the art. The solid obtained after drying can be further ground into a fine powder with various techniques known to the skilled in the art. The powder was passed through a 100 mesh screen in this example.

### Example 2

### Synthesis of Additional Polymeric Rheology Modifiers

Additional polymeric rheology modifiers were prepared following the basic procedure used to prepare Synthesis Example 1A. The compositions of these polymers and those of Synthesis Example 1 are summarized in the Table 2 below.

TABLE 2
Exemplary Polymeric Rheology Modifiers.

| | | | | | 2- | Iso C10 | | | t-BHP, |
|---|---|---|---|---|---|---|---|---|---|
| | IBOMA | Styrene | IBMA | LMA | EHMA | MA | X-Lnk′r | | 100% basis |
| Polymer # | Monomer Ratio (wt %)* | | | | | | | mg/kg | (pphm) |
| 1A | 50 | | 50 | | | | A | 500 | 0.0091 |
| 2.1 | | 40 | 50 | 10 | | | A | 105 | 0.0091 |
| 2.2 | | 40 | 50 | 10 | | | A | 213 | 0.0091 |
| 2.3 | | 40 | 50 | 10 | | | A | 410 | 0.0091 |
| 2.4 | | 40 | 50 | 10 | | | A | 851 | 0.0091 |
| 2.6 | 25 | 22 | 18 | 35 | | | A | 200 | 0.0091 |
| 2.7 | 25 | | 75 | | | | A | 500 | 0.0091 |
| 2.8 | 35 | | 65 | | | | A | 500 | 0.0091 |
| 2.9 | 35 | | 65 | | | | A | 500 | 0.0091 |
| 2.10 | 35 | | 65 | | | | A | 500 | 0.0073 |
| 2.11 | 35 | | 65 | | | | A | 650 | 0.0073 |
| 2.12 | 35 | | 65 | | | | A | 350 | 0.0073 |
| 2.13 | 35 | | 65 | | | | A | 650 | 0.0123 |
| 2.14 | 35 | | 65 | | | | A | 350 | 0.0123 |
| 2.15 | 35 | | 65 | | | | B | 1047 | 0.0073 |
| 2.16 | 35 | | 65 | | | | A | 502 | 0.0091 |
| 2.17 | 35 | | 65 | | | | B | 753 | 0.0098 |
| 2.18 | 35 | | 65 | | | | B | 758 | 0.0098 |
| 2.19 | 35 | | 65 | | | | B | 465 | 0.0098 |
| 2.20 | 35 | | 65 | | | | B | 1047 | 0.0123 |
| 2.21 | 35 | | 65 | | | | B | 465 | 0.0123 |
| 2.22 | 35 | | 65 | | | | A | 1500 | 0.0091 |
| 2.23 | 35 | | 65 | | | | A | 2000 | 0.0091 |
| 2.24 ^{c} | 35 | | 65 | | | | A | 3000 | 0.0091 |
| 2.25 | 50 | | 50 | | | | A | 204 | 0.0091 |
| 2.26 | 50 | | 50 | | | | A | 352 | 0.0091 |
| 2.27 | 50 | | 50 | | | | A | 500 | 0.0091 |
| 2.28 | 50 | | 50 | | | | A | 502 | 0.0065 |
| 2.29 | 35 | | 65 | | | | A | 500 | 0.0073 |
| 2.30 | 35 | | 65 | | | | A | 500 | 0.0073 |
| 2.31 | 50 | | 50 | | | | A | 1000 | 0.0091 |
| 2.32 | 50 | | 50 | | | | A | 2000 | 0.0091 |
| 2.33 | 50 | | 50 | | | | B | 218 | 0.0091 |
| 2.34 | 50 | | 50 | | | | B | 436 | 0.0091 |
| 2.35 | 50 | | 50 | | | | C | 218 | 0.0091 |
| 2.36 | 50 | | 50 | | | | C | 436 | 0.0091 |
| 2.37 | 75 | | 25 | | | | A | 500 | 0.0091 |
| 2.38 | | 10 | 90 | | | | A | 250 | 0.0091 |
| 2.39 | | 10 | 90 | | | | A | 500 | 0.0091 |
| 2.40 | | 10 | 90 | | | | A | 757 | 0.0091 |
| 2.41 | 4.98 | 13.02 | 82 | | | | A | 250 | 0.0091 |
| 2.42 | 4.98 | 13.02 | 82 | | | | A | 400 | 0.0091 |
| 2.43 | 4.98 | 13.02 | 82 | | | | A | 500 | 0.0091 |
| 2.44 | 4.98 | 13.02 | 82 | | | | A | 600 | 0.0091 |
| 2.45 | 4.98 | 13.02 | 82 | | | | A | 750 | 0.0091 |
| 2.46 | 9.41 | 24.59 | 66 | | | | A | 250 | 0.0091 |
| 2.47 | 9.41 | 24.59 | 66 | | | | A | 500 | 0.0091 |
| 2.48 | 9.41 | 24.59 | 66 | | | | A | 500 | 0.0091 |
| 2.49 | 9.41 | 24.59 | 66 | | | | A | 650 | 0.0123 |
| 2.50 | 9.41 | 24.59 | 66 | | | | A | 350 | 0.0123 |
| 2.51 | 9.41 | 24.59 | 66 | | | | A | 1000 | 0.0123 |
| 2.52 | 9.41 | 24.59 | 66 | | | | A | 350 | 0.0123 |
| 2.53 | 9.41 | 24.59 | 66 | | | | A | 650 | 0.0155 |
| 2.54 | 9.41 | 24.59 | 66 | | | | A | 650 | 0.0155 |
| 2.55 | 9.41 | 24.59 | 66 | | | | A | 1000 | 0.0187 |
| 2.56 | 9.41 | 24.59 | 66 | | | | A | 350 | 0.0187 |
| 2.57 | 50 | | 25 | 25 | | | A | 500 | 0.0091 |
| 2.58 | 30 | | 50 | | 20 | | A | 500 | 0.0091 |
| 2.60 | 65 | | | | | 35 | A | 504 | 0.0091 |
| 2.61 | 65 | | | | 35 | | A | 507 | 0.0091 |
| 2.62 ^{c} | 35 | | 65 | | | | | 0 | 0.0091 |
| 2.63** | 35 | | 65 | | | | A | 500 | 0.0091 |
| 2.64 ^{c} | 80 | | 20 | | | | | 0 | 0.0091 |
| 2.66 | 50 | | 50 | | | | A | 501 | 0.0091 |
| 2.67 | 9.41 | 24.59 | 66 | | | | A | 500 | 0.0091 |
| 2.68 | 9.41 | 24.59 | 66 | | | | B | 730.8 | 0.0155 |
| 2.69 ^{c} | 55 | 35 | 10 | | | | | 0 | 0.0091 |
| 2.70 ^{c} | | 40 | 50 | 10 | | | | 0 | 0.0091 |
| 2.71 ^{c} | 25 | 22 | 18 | 35 | | | | 0 | 0.0091 |
| 2.73 ^{c} | 50 | | 50 | | | | | 0 | 0.0091 |
| 2.75 ^{c} | | 10 | 90 | | | | | 0 | 0.0091 |
| 2.76 ^{c} | 4.98 | 13.02 | 82 | | | | | 0 | 0.0091 |
| 2.77 ^{c} | 9.41 | 24.59 | 66 | | | | | 0 | 0.0091 |
| 2.79 ^{c} | 65 | | | | 35 | | | 0 | 0.0091 |
| 2.80 ^{c} | 65 | | | | | 35 | | 0 | 0.0091 |
| 2.81*** | 35 | | 65 | | | | | | |

^{c} Comparative Examples
*Percentage of monomer as a mass percent of the total amount of monomer
**Sample was spray dried using a Mobile Minor Spray Dryer from GEA with a rotary atomizer AF-05-A, made by Costruzioni Meccaniche Turbine with inlet temperature 145° C. and outlet temperature 78° C.
***Sample made using the process of example 1B.
IBOMA = isobornyl methacrylate;
IBMA = isobutyl methacrylate;
LMA = lauryl methacrylate;
SMA = stearyl methacrylate;
2-EH MA = 2-ethylhexyl methacrylate;
IsoC10 MA = isodecyl methacrylate;
Crosslinker “A” is 1,6-hexanediol diacrylate (1,6-HDDA);
Crosslinker “B” is trimethylolpropane triacrylate (TMPTA);
Crosslinker C is pentaerythritol allyl ether (PEAE);
t-BHP = t-butyl hydroperoxide.

The oven dried powders were ground and passed through a 60 or 100 mesh screen before use in the following experiments. Powder can also be used without passing the 60-100 mesh screen but it would take longer time to dissolve.

### Example 3

### Polymeric Rheology Modifier Thickening Performance in Fatty Acid Esters

Throughout the examples, if not specifically noted, a clear and thickened oil can be obtained with any order of addition at room temperature with shaking by hands or a machine. However, it is advisable to add the powder thickener of the disclosure to a pre-blended liquid oil last to avoid potentially long dissolution time of the powder into the liquid. If an overhead mixer is used, slowly add the thickener last. The complete dissolution to a homogeneous solution (visually no more individually swollen particles) depends on the particle size of the powder. We noticed that if the powder was passed through a 100-mesh screen, the powder would usually dissolve within a few minutes to 20 minutes at room temperature. All samples were mixed until all oil thickener particles were dissolved and the samples were homogeneous. As known in the art, heating would increase the dissolution rate of powders in liquids.

The viscosity of exemplary polymeric rheology modifiers of Table 2 in soy methyl ester (SME) was measured. SME is a clear low viscosity liquid with a viscosity of about 3-8 cps at room temperature. Increased viscosity is indicative of thickening ability of the rheology modifier being analysed. The SME used was from Cargill (trade name Methyl soyate), Enviro Saver (trade name soy methyl ester), or Chempoint (trade name SoyGold 1000). The source of SME did not cause significant difference in results.

To prepare the samples, the polymeric rheology modifiers were dispersed slowly into SME under adequate agitation and were dissolved fully with agitation before the viscosity measurement. Viscosity of the thickened oil samples above was measured with a Brookfield DV-1 Prime viscometer on a sample within a glass jar having a 2 inch inner diameter using a standard Brookfield viscometer process well known to those skilled in the art. Except where noted otherwise in Table 3, viscosities for the samples in this Example 3 were measured at about 22° C. with a #3 spindle.

TABLE 3
Thickened SME Oil with Exemplary Polymeric Rheology Modifiers

| | | Polymeric | Polymeric | Brookfield Viscosity, cps | | | | Viscosity |
|---|---|---|---|---|---|---|---|---|
| | SME, | Rheology | Rheology | 10 | 20 | 50 | 100 | Ratio of |
| Sample # | wt % | Modifier # | Modifier, wt % | rpm | rpm | rpm | rpm | 10:100 rpm |
| 3.1 | 97 | 1 | 3 | 640 | 440 | 288 | 216 | 2.96** |
| 3.2 | 97 | 2.7 | 3 | 560 | 360 | 248 | 188 | 2.98** |
| 3.3 | 97 | 2.37 | 3 | 360 | 260 | 200 | 156 | 2.31** |
| 3.4 | 97 | 2.26 | 3 | 600 | 440 | 304 | 240 | 2.50** |
| 3.5 | 97 | 2.28 | 3 | 510 | 365 | 244 | 188 | 2.71* |
| 3.6 | 97 | 2.27 | 3 | 450 | 320 | 208 | 158 | 2.85* |
| 3.7 | 97 | 2.16 | 3 | 330 | 245 | 170 | 134 | 2.46 |
| 3.8 | 97 | 2.8 | 3 | 360 | 265 | 184 | 145 | 2.48 |
| 3.9 | 97 | 2.11 | 3 | 240 | 185 | 132 | 109 | 2.20 |
| 3.10 | 97 | 2.12 | 3 | 630 | 460 | 318 | 248 | 2.54 |
| 3.11 | 95.8 | 2.13 | 4.2 | 1230 | 875 | 568 | 379 | 3.25 |
| 3.12 | 97 | 2.21 | 3 | 260 | 220 | 145 | 115 | 2.26 |
| 3.13 | 96.4 | 2.18 | 3.6 | 360 | 260 | 170 | 130 | 2.77 |
| 3.14 | 96.4 | 2.17 | 3.6 | 350 | 255 | 168 | 125 | 2.80 |
| 3.15 | 95.8 | 2.15 | 4.2 | 570 | 400 | 262 | 189 | 3.02 |
| 3.16 | 95.8 | 2.19 | 4.2 | 1190 | 850 | 556 | 412 | 2.89 |
| 3.17 | 95.8 | 2.20 | 4.2 | 700 | 475 | 300 | 218 | 3.21 |
| 3.18 | 97 | 2.29 | 3 | 480 | 350 | 230 | 177 | 2.71 |
| 3.19 | 97 | 2.50 | 3 | 400 | 325 | 246 | 197 | 2.03 |
| 3.20 | 97 | 2.52 | 3 | 480 | 370 | 272 | 215 | 2.23 |
| 3.21 | 97 | 2.53 | 3 | 270 | 225 | 162 | 130 | 2.08 |
| 3.22 | 97 | 2.54 | 3 | 260 | 205 | 148 | 119 | 2.18 |
| 3.23 | 97 | 2.56 | 3 | 400 | 305 | 222 | 172 | 2.33 |
| 3.24 | 97 | 2.47 | 3 | 550 | 380 | 290 | 204 | 2.70 |
| 3.25 | 97 | 2.2 | 3 | 520 | 420 | 296 | 232 | 2.24** |
| 3.26 | 97 | 2.3 | 3 | 280 | 220 | 160 | 120 | 2.33** |
| 3.27*** | 97 | 2.81 | 3 | 540 | 420 | 312 | 259 | 2.09 |

*Viscosity measured 1 day after preparation at 27.5° C.
**Viscosity measured with a #4 spindle.
***Sample prepared by method of Example 1B

The results in this Table 3 show that the rheology modifiers shown in Example 3 are able to thicken SME and that the thickened oil compositions display shear thinning property (e.g. viscosity is lower at higher shear rate). As used herein, shear-thinning refers to non-Newtonian fluids which have decreased viscosity when subjected to shear strain. For the purposes of the current disclosure, formulations have strong shear thinning property if the viscosity ratio of the formulation at 10 rpm and 100 rpm is greater than 2 as measured by a Brookfield viscometer. High shear thinning property is an advantageous property and is believed to be associated with the ability of the polymeric rheology modifier to suspend solids.

### Example 4

### Additional Thickening Properties in Oils

### Example 4A

### Viscosity measurements

Samples were prepared in accordance with the method of Example 3. The viscosities of a thickened oil composition containing soy methyl ester and soy oil (PMC Biogenix) in this Example were measured with a Brookfield DV-II+ viscometer with spindle setting #64 at 22° C. Soy oil is a clear liquid with a viscosity of about 45-50 cps at room temperature.

TABLE 4A
Thickened Oils (SME and Soybean Oil) with Exemplary
Polymeric Rheology Modifiers of Table 2

| Brookfield DV-II+ Pro spindle # 64. | | | | | | | | |
|---|---|---|---|---|---|---|---|---|
| | | | Polymeric | | | | | |
| | | | rheology | | Viscosity (cps). 3 days at RT | | | |
| | SME | Soy oil | modifier# | Comment | 100 | 50 | 20 | 10 |
| Sample | wt % | wt % | (4.76%) | (visual) | rpm | rpm | rpm | rpm |
| 4.1 | 95.24 | | 2.25 | Clear. Short | 1120 | 1392 | 1950 | 2300 |
| | | | | hydration time. | | | | |
| | | | | Fish eyes may | | | | |
| | | | | occur if mixing is | | | | |
| | | | | not adequate. | | | | |
| 4.2 | 95.24 | | 1 | Clear appearance. | 700 | 900 | 1650 | 2500 |
| | | | | Short hydration | | | | |
| | | | | time. | | | | |
| 4.3 | 47.62 | 47.62 | 2.25 | Clear. Short | 1700 | 2100 | 2790 | 3659 |
| | | | | hydration time. | | | | |
| 4.4 | 47.62 | 47.62 | 1 | Clear. Short | | | | |
| | | | | hydration time. | | | | |
| | | | | Thickened | | | | |
| 4.5 | 14.29 | 80.95 | 2.25 | 1 hour to create | 3000 | 3479 | 4350 | 5300 |
| | | | | clear appearance. | | | | |
| 4.6 | 14.29 | 80.95 | 1 | 1 hour to create | | | | |
| | | | | clear appearance. | | | | |
| | | | | Thickened | | | | |
| 4.7 | 23.81 | 71.43 | 1 | 30 mins to create | | | | |
| | | | | clear appearance. | | | | |
| | | | | Thickened | | | | |

Table 4A shows that the rheology modifiers of the present disclosure can thicken mixtures of SME and soybean oil.

### Example 4B

### Thickening of Various Oils with Thickeners of the Present Invention

The thickening ability of various thickeners of the present disclosure in various oils was studied. The studied samples contained 5% thickener and 95% oils. Each sample was prepared by adding 5% thickener to 95% oil and immediately mixed vigorously for about 15-30 minutes. Samples 4.8-4.22 and 4.32 of Table 4B were prepared at room temperature. Samples 4.23-4.31 of Table 4B were prepared at ˜70° C. for 15-30 minutes. Samples were placed at room temperature overnight and were shaken by hand before observation to generate air bubbles. Appearance of each sample at room temperature and thickening of each were observed and recorded in Table 4B. The thickening was determined qualitatively and it could be easily compared to the original organic liquid visually. The thickening was observed by looking at how long the bubbles remained suspended in the liquid after shaking. Generally, the bubbles rise to the surface quickly (less than ˜10 seconds) in pure oils after shaking because the viscosity of the oils are low (typically <100 mPas). If thickening occurs, the bubbles in a thickened oil tend to remain suspended in the liquid for at least more than a few minutes, and in some cases at least 15 minutes longer than the bubbles in the pure oil. All samples in Table 4B were able to trap bubbles for more than 15 minutes longer than in the respective pure oils except where indicated.

TABLE 4B
Composition of example 4 - thickening of 95 wt % oils with 5 wt % thickener

| Smpl | Thickener | Oil | Chemical class | Appr at RT |
|---|---|---|---|---|
| 4.8 | 2.63 | Soy methyl ester | Ester | Clear liq |
| 4.9 | 2.63 | ISPM (isopropyl | Ester | Clear liq, sl. |
| | | myristate) | | purple |
| 4.10 | 2.63 | Finsolv ® TN | Ester | Clear liq, very |
| | | (C12/15 benzoate) | | viscous |
| 4.11 | 2.63 | Ethylene glycol butyl | Alkylene glycol C1-C8 | Clear |
| | | ether acetate | ether acetate | |
| 4.12 | 2.63 | Diethylene glycol butyl | Alkylene glycol C1-C8 | Clear |
| | | ether acetate | ether acetate | |
| 4.13 | 2.63 | 2-Butoxyethanol acetate | Ester | Clear |
| 4.14 | 2.63 | UV cocktail | Organic sunscreen Al | Clear Gel |
| 4.15 | 2.63 | Neo Heliopan ® 303 | Organic sunscreen Al | Clear gel |
| | | (octocrylene) | | |
| 4.16 | 2.63 | Neo Heliopan ® HMS | Organic sunscreen Al | Clear gel |
| | | (homosalate) | | |
| 4.17 | 2.63 | Neo Heliopan ® OS | Organic sunscreen Al | Clear gel |
| | | (ethylhexyl salicylate) | | |
| 4.18 | 2.63 | Neo Heliopan ® MA | Organic sunscreen Al | Clear Gel |
| | | (menthyl anthranilate) | | |
| 4.19 | 2.63 | Neo Heliopan ® AV | Organic sunscreen Al | Clear liq, very |
| | | (ethylhexyl | | viscous |
| | | methoxycinnamate) | | |
| 4.20 | 2.66 | UV cocktail* | Organic sunscreen Al | Clear Gel |
| 4.21 | 2.63 | Dow Corning ® 556 F | Silicone oil | Clear liq, very |
| | | | | viscous |
| 4.22 | 2.63 | Dow Corning ® 550 Fluid | Silicone oil with | Clear |
| | | | aromatic group | |
| 4.23 | 2.63 | Soybean oil | Triglyceride | Clear liq |
| 4.24 | 2.63 | Estol 3609 (glycerol tri- | Triglyceride | Clear liq |
| | | 2-ethylhexanoate ester | | |
| | | oil) | | |
| 4.25 | 2.63 | Prisorine 2041 glycerol | Triglyceride | Translucent |
| | | triisostearate | | liquid |
| 4.26 | 2.63 | Corn oil | Triglyceride | Clear |
| 4.27 | 2.63 | Soybean oil | Triglyceride | Clear |
| 4.28 | 2.63 | Rapeseed oil | Triglyceride | Clear |
| 4.29 | 2.63 | Canola oil | Triglyceride | Clear |
| 4.30 | 2.63 | Linseed oil | Triglyceride | Clear |
| 4.31 | 2.63 | Macadamia oil | Triglyceride | Clear |
| 4.32 | 2.63 | Caprylic capryl | Triglyceride | Clear |
| | | triglyceride (Myritol 318) | | |
| 4.33 | 2.63 | Butyloctyl Salicylate | Aromatic compound | Clear |
| | | (Hallbrite BHB) | | |
| 4.34 | 2.25 | Methyl benzoate | Ester | Clear |
| 4.35 | 2.58 | Methyl benzoate | Ester | Clear |
| 4.36 | 2.64 | Methyl benzoate | Ester | Clear, |
| | | | | Trapped bubbles |
| | | | | for ~15 mins |
| 4.37 | 2.55 | Methyl benzoate | Ester | Clear |
| 4.38 | 2.57 | Methyl benzoate | Ester | Clear |
| 4.39 | 2.63 | Methyl benzoate | Ester | Clear |
| 4.40 | 2.63 | Acetyl tributyl citrate | Ester | Clear |
| 4.41 | 2.63 | Butyryltri-n-hexyl citrate | Ester | Clear |
| 4.42 | 2.63 | Tributyl citrate | Ester | Clear |
| 4.43 | 2.63 | methyl cocoate | Ester | Clear |
| 4.44 | 2.63 | 2-ethylhexyl lactate | Ester | Clear |
| 4.45 | 2.25 | Butyl lactate | Ester | Clear |
| 4.46 | 2.64 | Butyl lactate | Ester | Clear. |
| | | | | Trapped bubbles |
| | | | | for <15 mins |
| 4.47 | 2.43 | Butyl lactate | Ester | Clear |
| 4.48 | 2.12 | Butyl lactate | Ester | Clear |
| 4.49 | 2.20 | Butyl lactate | Ester | Clear |
| 4.50 | 2.12 | Butyl lactate | Ester | Clear |
| 4.51 | 2.55 | Butyl lactate | Ester | Clear |
| 4.52 | 2.56 | Butyl lactate | Ester | Clear |
| 4.53 | 2.24 | Butyl lactate | Ester | Hazy. No |
| | | | | thickening |
| 4.54 | 2.58 | Butyl lactate | Ester | Clear |
| 4.55 | 2.63 | Propyl lactate | Ester | Clear |
| 4.56 | 2.63 | Dimethyl adipate | Ester | Clear |
| 4.57 | 2.63 | Diethyl maleate | Ester | Clear |
| 4.58 | 2.63 | Methyl oleate | Ester | Clear |
| 4.59 | 2.63 | Diethyl succinate | Ester | Clear |
| 4.60 | 2.63 | Methyl ester of C810 | Ester | Clear |
| | | acid | | |

*UV cocktail: Avobenzone 9.37 wt %, Homosalate 40.62 wt %, Octisalate 15.63 wt %, Octocrylene 15.63 wt %, Oxybenzone 18.75 wt %

It can be seen from the results shown in Table 4B that the thickeners of the present disclosure can thicken various oils useful in personal care formulations and in sunscreen formulations in particular. Comparative samples containing no cross-linker (2.64) and samples containing a very high level of cross-linker (2.24) showed limited bubble suspension ability.

### Example 5

### Thickened Clear Organic Sunscreen Oil

An organic sunscreen oil was prepared by adding thickener 2.63 into a pre-blended mixture of a UV cocktail (used in example 4) and isopropyl myristate (ISPM). The sample was hand shaken immediately for a few minutes. The sample became thickened and turn clear quickly. The composition, viscosity and appearance were shown in Table 5.

TABLE 5
Thickened clear organic sunscreen oil

| Sample | #5.1 | |
|---|---|---|
| UV-cocktail | 32 | wt % |
| Thickener 2.63 | 4 | wt % |
| ISPM | 64 | wt % |
| Viscosity, initial (1-4 RV5 | 1360 | cps |
| Spindle, 5-6 RV6) 10 rpm | | |
| Appearance, initial | Clear brownish, suspended bubbles | |
| Viscosity after 4 wks at 50° C. | 1240 | cps |
| (1-4 RV5 Spindle, 5-6 RV6) 10 rpm | | |

| | |
|---|---|
| Appearance, after 4 wks at 50° C. | Clear brownish liquid, stable |
| Stability after 11 weeks | Stable, no separation |

### Example 6

### Comparison of a Thickened Oil by Thickener 2.63 with Johnson's® Baby Oil Gel

Skin oils were prepared by adding thickener 2.63 into a pre-blended liquid mixture. The samples were hand shaken immediately for a few minutes. The sample thickened and turned clear quickly. The composition, viscosity and appearance were shown in Table 6.

TABLE 6
Comparison of a thickened oil by thickener 2.63 with Johnson's ® Baby Oil Gel

| | #6.1 | | | |
|---|---|---|---|---|
| Sample | (comparative) | #6.2 | #6.3 | #6.4 |
| Johnson's ® | 100% | | | |
| baby oil gel | | | | |
| ISPM | | 55 wt % | 46.65 | 46 |
| Soy | | 31.2 wt % | 46.75 | 46 |
| Thickener 2.63 | | 4.6 wt % | 4.93 | 8 |
| Eucalyptus oil | | 9.2 wt % | 1.67 | |
| Comment | Clear. Easy to rub. | Clear. Easy to rub. | Clear. Easy to rub. | Clear. Easy to rub. |
| | Suspend big | Suspend small | Suspend small | Suspend small |
| | bubbles. Greasy and | bubbles for >30 | bubbles for >30 | bubbles >1 week. |
| | tacky after-feel | mins. Non-tacky | mins. Non-tacky | Similar greasy and |
| | | non-greasy | non-greasy | tacky feel to |
| | | | | Johnson's baby oil |
| | | | | gel. |

Johnson's® baby oil gel is a well-known product in the market place. However, it uses mineral oil as the skin emollient. It feels greasy after rubbing by hand.

It shows that thickener 2.63 can thicken plant derived oils (ISPM and soy oil) and the thickened oil can have at least similar after feel when compared to Johnson's® baby oil gel.

### Example 7

### Thickened Personal Care Sanitizer Formulations

Ethanol and isopropyl alcohol are well known sanitizers. Ethanol sanitizer formulations are known comprising ethanol, water, and Carbopol® thickener. Isopropyl alcohol is a more effective antimicrobial than ethanol; however, it cannot be thickened effectively. There is no thickened isopropyl alcohol hand sanitizer on the market.

This example is to show that it is possible to thicken isopropyl alcohol and ethanol without the use of water (or Carbopol) by the rheology modifiers, if a suitable oil is present.

The data in Table 7 demonstrate that the thickeners of the disclosure can thicken mixture of ethanol or isopropyl alcohol with an organic liquid suitable for use in a personal care sanitizer formulation. Moreover, incorporating an oil in a hand sanitizer can reduce the “dry-hand” effect, and provide an improved aesthetic performance for the user.

The samples were prepared by mixing the liquids first, followed by adding the thickener 2.63 with further mixing until all solid particles dissolved.

TABLE 7
Thickened Personal Care Sanitizer Formulations

| Example | Thickener | | | | Organic | | |
|---|---|---|---|---|---|---|---|
| # | # | Wt % | Alcohol | Wt % | liquid | Wt % | |
| 7.1 | 2.63 | 5 | Ethanol | 46.8 | SME | 48.2 | Purple/green. >1 |
| | | | | | | | hr bubbles |
| 7.2 | 2.63 | 5.000 | Ethanol | 47.50 | d-limonene | 47.50 | Purple/green. >1 |
| | | | | | | | hr bubbles |
| 7.3 | 2.63 | 5.000 | IPA* | 70.00 | Soy methyl | 25.00 | Purple/green. >1 |
| | | | | | ester | | hr bubbles |
| 7.4 | 2.63 | 5.000 | Ethanol | 47.50 | Soy oil | 47.50 | Milky, viscous |
| 7.5 | 2.63 | 5.000 | Isopropyl | 47.50 | Ethyl | 47.50 | Purple/green. >1 |
| | | | alcohol | | benzoate | | hr bubbles |
| 7.6 | 2.63 | 10.000 | Ethanol | 45.00 | Pine oil | 45.00 | Green. Very |
| | | | | | (60%) | | viscous. |
| | | | | | | | Surface didn't |
| | | | | | | | level after 1 hr. |
| 7.7 | 2.63 | 10.4 | Ethanol | 44.8 | Peppermint | 44.8 | Green. Very |
| | | | | | oil | | viscous. |
| | | | | | | | Surface didn't |
| | | | | | | | level after 1 hr. |
| 7.8 | 2.63 | 13.3 | Ethanol | 60 | d-limonene | 26.7 | Green. Viscous. |
| 7.9 | 2.63 | 10.000 | Ethanol | 45.00 | d-limonene | 45.00 | Green. Very |
| | | | | | | | viscous. |
| | | | | | | | Surface didn't |
| | | | | | | | level after 1 hr. |
| 7.10^{c} | 2.63 | 5 | Ethanol | 95 | | | Not soluble. |
| | | | | | | | No thickening. |
| 7.11^{c} | 2.63 | 5 | Isopropyl | 95 | | | Greenish and |
| | | | alcohol | | | | thickened only |
| | | | | | | | at >~40 C. |
| | | | | | | | Hazy at room |
| | | | | | | | temp. |

*IPA is isopropyl alcohol
^{c}comparative example

### Example 8

### Thickened Personal Care Sanitizer Formulation Evaporation Study

The comparative data of Table 8 demonstrate that thickened sanitizer formulations as disclosed herein not only have a reduced risk for spillage but also have a slower evaporation rate, hence, a prolonged antimicrobial time on the skin.

TABLE 8
Thickened Skin Sanitizer with Isopropyl
Alcohol and Skin Emollient

| | 8.1 | |
|---|---|---|
| Sample | (comparative) | #8.2 |
| Isopropyl alcohol (Skin Sanitizer) | 50 wt % | 45% |
| Soy methyl ester (Skin Emollient) | 50 wt % | 45% |
| thickener 2.63 | | 10% |
| Appearance | Clear, low | Clear. Stable. |
| | viscosity | Thickened |

| | | |
|---|---|---|
| Time, minutes | wt % loss | wt % loss |

Weight % loss in a 9 gram sample in open air at room temperature:

| | | |
|---|---|---|
| 0 | 0 | 0 |
| 5 | 3.2 | 1.0 |
| 10 | 6.8 | 2.5 |
| 20 | 10.9 | 4.1 |
| 25 | 13.3 | 5.6 |
| 30 | 14.9 | 6.2 |
| 40 | 18.1 | 7.6 |
| 60 | 26.2 | 10.3 |
| 70 | 33.3 | 11.3 |
| 80 | 36.3 | 12.2 |

Examples 3-8 are examples of organic liquid formulations thickened by the oil thickeners of the present invention. These thickened organic liquid formulations are useful for personal care applications. These thickened organic liquids are useful either as polymeric emulsifiers in formulating surfactant-free W/O emulsions, stabilizers for surfactant-free O/W emulsions, or liquid carriers to suspend solids such as metal oxides or other particulates.

### Example 9

### Surfactant-Free Emulsions—Ability of a Thickened Oil to Trap Water Droplets Forming Water-In-Oil (W/o) Emulsions

The following samples were prepared by hand shaking for a few minutes. For sample 9.2 the thickener (2.63) was mixed with the oil (ISPM) first until fully dissolved. A clear and thickened oil was obtained. Water was then added to the thickened oil and mixed until a white, smooth emulsion was obtained. Samples 9.1 and 9.2 are water-in-oil emulsions. Strictly speaking, an emulsion is defined as a liquid composition containing water, at least one oil, and at least a surfactant emulsifier. However, sample 9.2 is an emulsion without the traditional surfactant emulsifiers.

TABLE 9
Ability of a thickened oil to trap water droplets

| | #9.1 | |
|---|---|---|
| Sample | (comparative) | #9.2 |
| Thickener 2.63 | | 8 |
| ISPM | | 72 |
| Water | 20 wt % | 20 |
| Johnson's ® | 80 wt % | |
| baby oil gel | | |
| Stability | Separate 1 day at | Stable. No separation |
| | room temperature | 11 wks at 50° C. |
| Comment | Unable to | Smooth. White viscous liquid. |
| | emulsify water | Lotion-like. Viscosity 5340 cps |
| | | (10 RPM) by Brookfield |
| | | DV-II+ spdl #3 |

Johnson's® baby oil gel (a thickened oil) has many air bubbles in it. However, the Johnson's baby oil gel does not have the ability to hold water in a stable emulsion while the sample 9.2 with thickener 2.63 can hold water very well. This shows that not all thickened oils (or even gelled oil such as the Johnson's baby oil gel) can trap water.

### Example 10

### Surfactant-Free Emulsions—Ability of Thickened Oils to Trap Water Droplets Forming Water-In-Oil (W/O) Emulsions—More Examples

TABLE 10
Surfactant-free emulsions - Ability of thickened oils to
trap water droplets forming water-in-oil (W/O) emulsions

| | | | Thickener | Water | |
|---|---|---|---|---|---|
| Sample | Oil | Thickener | wt % | wt % | Appearance |
| #10.1 | Ethyl benzoate | #2.63 | 3.6% | 28.6% | White emulsion. Viscosity |
| | 67.8% | | | | 4343 cps by DV-II+, |
| | | | | | spdl 3, 10 rpm |
| #10.2 | Ethyl benzoate | #2.14 | 4% | 20% | White emulsion. Low |
| | 64% + ISPM 12% | | | | viscosity |
| #10.3 | Ethyl benzoate | #2.13 | 6% | 20% | White emulsion. Low |
| | 62% + ISPM 12% | | | | viscosity |
| #10.4 | Ethyl benzoate | #2.49 | 6% | 20% | White emulsion. Low |
| | 62% + ISPM 12% | | | | viscosity |
| #10.5 | Ethyl benzoate | #2.55 | 6% | 20% | White emulsion. Low |
| | 62% + ISPM 12% | | | | viscosity |
| #10.6 | Ethyl benzoate | #2.27 | 6% | 20% | White emulsion. Low |
| | 62% + ISPM 12% | | | | viscosity |
| #10.7 | Ethyl benzoate | #2.58 | 6% | 20% | White viscous emulsion. |
| | 62% + ISPM 12% | | | | |
| #10.8 | Ethyl benzoate | #2.15 | 6% | 20% | White emulsion. Low |
| | 40% + ISPM 34% | | | | viscosity |
| #10.9 | Ethyl benzoate | #2.57 | 6% | 20% | White emulsion. Low |
| | 62% + ISPM 12% | | | | viscosity |

All samples were prepared using the same method as in sample #9.2. All samples in Table 10 were stable (no visible separation) after 2 weeks at 54° C.

### Example 11

### Stabilized Surfactant-Free Water-Thickened O/W Emulsions

Pemulen™ TR-1 and TR-2, both acrylates/C10-30 alkyl acrylate crosspolymers by Lubrizol, are well-known polymeric thickeners capable of trapping (“emulsifying”) oil droplets in water to form O/W emulsions. Ganex V-220, PVP (and) VP/Eicosene Copolymer, is able to thicken a blend of organic sunscreen active ingredients (the UV cocktail in example 5) it also can function as a film former in sunscreen emulsions.

In this example, we studied the effect of thickener 2.63 and Ganex V-220, respectively, on the stability of O/W emulsions wherein the emulsion is formed with acrylates/C10-30 alkyl acrylate crosspolymer. The results are shown in Table 11, in which all quantities are given in wt %.

TABLE 11
Surfactant-free O/W emulsions stabilized by Acrylates/C10-30 Alkyl Acrylate Crosspolymer

| | #11.1 | #11.2 | | #11.4 | |
|---|---|---|---|---|---|
| Sample | (comparative) | (comparative) | #11.3 | (comparative) | #11.5 |
| Thickened water | 68 | 66 | 66 | | |
| (0.6% Pemulen TR-2, | | | | | |
| pH 6.8 by NaOH) | | | | | |
| Thickened water | | | | 70 | 68 |
| (0.5% Pemulen TR-1, | | | | | |
| pH ~4.5) | | | | | |
| UV cocktail | 32 | 32 | 32 | | |
| (Example 4) | | | | | |
| Thickener 2.63 | | | 2 | | 2 |
| Ganex V-220 | | 2 | | | |
| Finsolv ® TN | | | | 30 | 30 |
| (C12-15 benzoate) | | | | | |
| Preparation | Room temp | ~50° C. | Room | Room temp | ~80° C. |
| condition | | | temp | | |
| Stability | Separated at | Separated 1 day | Stable 18 days | Separated at | Stable 2 weeks |
| | room temp | at 50° C. | at 50° C. | room temp | at 50° C. |
| Appearance at | Emulsion | Grainy paste | Thickened | Emulsion | Smooth white |
| room temp | separated | | emulsion | separated | cream |
| Viscosity by | | | | | 24000 |
| Brookfiled DV-II+ | | | | | |
| with RV7 Spindle, | | | | | |
| 10 rpm | | | | | |

In preparing the samples 11.2-11.5, the powder (thickener 2.63 or Ganex V-220) was added to the oil phase (the UV cocktail or Finsolv TN) at the indicated temperature in an overhead mixer until all powder particles dissolved, then the aqueous phase was added to the oil phase and mixed until the sample was homogeneous.

The results in Table 11, samples 11.1 and #11.4, showed that UV cocktail oil droplets and C12-15 benzoate oil droplets were not stable in the thickened waters (by acrylates/C10-30 alkyl acrylate crosspolymer). Sample 11.2 showed that even in the presence of 2% Ganex V-220, the UV cocktail oil droplets were not stable in the thickened water by acrylates/C10-30 alkyl acrylate crosspolymer. However, the UV cocktail oil droplets and C12-15 benzoate oil droplets each were very stable when 2% thickener 2.63 was present, forming stable O/W emulsions, as shown in samples 11.3 and 11.5, respectively.

Sample 11.3 can be used as a sunscreen personal care formulation and sample 11.5 can be used as a skin oil personal care formulation.

### Example 12

### Surfactant-Free W/O Sunscreen Emulsions with Exemplary Thickener 2.63

The following samples were prepared by adding thickener 2.63 to the oil phase to form the oil phase first. The emulsions were formed by adding the aqueous phase to the oil phase after all the thickener powder dissolved and the oil phase became clear. The results are shown in table 12, in which all quantities are given in wt %.

TABLE 12
Surfactant-free W/O sunscreen emulsions with exemplary thickener 2.63

| Sample | #12.1 | #12.2 | #12.3 | #12.4 |
|---|---|---|---|---|
| Finsolv ® TN | 36 | 36 | | |
| (C12-15 benzoate) | | | | |
| UV Cocktail | 33.1 | 33.1 | 22.4 | 32 |
| (Example 4) | | | | |
| Thickener 2.63 | 2.3 | 2.3 | 2 | 1.5 |
| Water | 28.6 | | | |
| 0.4% Pemulen EZ-4U | | 28.6 | | |
| (pH 5.5 w/NaOH) | | | | |
| Thickened water | | | 53.2 | |
| (0.3% Pemulen TR2, | | | | |
| pH ~7 with NaOH) | | | | |
| Thickened water | | | | 66.5 |
| (0.285% Pemulen TR2, | | | | |
| pH 6.6 by NaOH) | | | | |
| ISPM | | | 22.4 | |
| Stability | Stable 2 wks | Stable 2 wks | Stable 2 wks | Stable 3 months |
| | at 50° C. | at 50° C. | at 50° C. | at 50° C. |
| Appearance at room temp | Viscous | Viscous | Smooth white | Smooth white |
| | emulsion | emulsion | viscous emulsion | viscous emulsion |

### Example 13

### Metal Oxide Suspension in Surfactant-Free Emulsions

The sample #13.1 was prepared first with hand shaking. It separated into a two-phase system after about 30 minutes. The sample #13.2 was prepared by adding thickener 2.63 to sample # 13.1 and shaken until all powder particles dissolved. Part of the sample #13.2 was used to prepare the sample #13.3 (by adding TiO2 to the sample #13.2 with shaking).

Sample #13.4 was prepared by adding thickener 2.63 to the oils to form the oil phase first, followed by adding the aqueous phase to the oil phase after all the powder dissolved and the oil phase became clear. TiO2 was added last to the sample. Sample was shaken at each step.

Table 13. Metal oxide suspension in surfactant-free emulsions with exemplary thickener 2.63

| | #13.1 | | | |
|---|---|---|---|---|
| Sample | (Comparative) | #13.2 | #13.3 | #13.4 |
| Formulation type | Water and oil | Surfactant-free | TiO2 suspension | TiO2 suspension |
| | mixture | W/O emulsion | in surfactant-free | in surfactant-free |
| | | | W/O emulsion | W/O sunscreen emulsion |
| Water | 20 parts | 20 parts | 20 parts | |
| 0.3% Pemulen TR2 | | | | 41.2 wt % |
| Finsolv ® TN | 75 parts | 75 parts | 75 parts | |
| (C12-15 benzoate) | | | | |
| Isopropyl myristate | | | | 22.4 wt % |
| UV cocktail | | | | 22.4 wt % |
| (example 4) | | | | |
| Thickener 2.63 | | 5 parts | 5 parts | 4 wt % |
| UV Titan M-111 | | | 10 parts | 10 wt % |
| titanium dioxide | | | | |
| Stability | Separated | Stable 2 wks | Stable 2 wks | Stable 2 wks |
| | within ~30 | at 50° C. | at 50° C. | at 50° C. |
| | minutes at | | | |
| | room temp | | | |
| Appearance | 2 phase sample | White smooth | White | White |
| | | emulsion. Low | suspension. | suspension. |
| | | viscosity | Low viscosity | Viscous |

Sample 13.2 shows again that thickener 2.63 is able to form stable surfactant-free W/O emulsion. Samples 13.3 and 13.4 show that the surfactant-free emulsions can suspend solid particles (TiO2).

### Example 14

### SPF Enhancing Effect

All SPF measurements were made in vitro using a UV-1000S Ultraviolet Transmittance Analyzer, available from Labsphere, and VITRO-SKIN® synthetic skin substitute, available from IMS Inc. To begin, the VITRO-SKIN® synthetic skin substitute was hydrated for 16 hours in a controlled humidity chamber per the protocol suggested by the manufacturer. For each sample, a 6.2 cm×8.0 cm section of VITRO-SKIN® was cut. The VITRO-SKIN® was placed rough side up on a foam block used to simulate the flexibility of human skin and a product dose of 2 μL/cm² (approximately 100 μL) of the sunscreen formulation was applied using a micropipettor evenly across the VITRO-SKIN® surface. The sunscreen formulation was gently rubbed in with a gloved finger for 20 to 30 seconds, mimicking the application of sunscreen to a body. Once the sunscreen was spread on the skin, the skin was sandwiched between two 6 cm×6 cm frames (or glassless slides) and allowed to dry for 20 minutes. SPF measurements were made using the UV-1000S Ultraviolet Transmittance Analyzer. Five SPF values were taken at different locations on each skin covered with the sample. The results are shown in Table 14, in which the quantities of each of the components in each formulation are stated in wt %.

TABLE 14
SPF enhancing effect of thickener 2.63

| | #14.1 | | #14.3 | |
|---|---|---|---|---|
| Sample | (comparative) | #14.2 | (=sample #12.2)* | #14.4* |
| SME | 68 | 64% | | |
| Thickener 2.63 | | 4% | 2.3% | 2% |
| UV cocktail | 32 | 32% | 33.1% | 20% |
| Finsolv ® TN | | | 36% | 25% |
| Water | | | 28.6% | |
| 0.4% Pemulen | | | | 53% |
| EZ-4U (pH 5.5) | | | | |
| Number of data | 30 | 40 | 25 | 25 |
| points | | | | |
| Average SPF | 51 | 89 | 91 | 39 |
| Std dev | 9 | 13 | 20 | 13 |

*SPF was measured after 2 weeks at 50° C.

Comparing samples #14.1 and #14.2, it showed that thickener 2.63 was able to enhance the SPF of sunscreen actives.

Without wishing being bound to any theory, it is believed that a thickened product (sample 14.2) forms a uniform film on top of the rough skin surface while a thinner sample (sample 14.1) sinks to valleys of the rough skin surface. In addition, a thickened sample evaporates slower than a thinner sample.

### Example 15

### SPF and Water Resistance Study

The exemplary formulation was compared to three comparative examples as shown in Table 15. The labels on the commercial products (Neutrogena® Pure and Free™ Baby Sunscreen Lotion (SPF 60) and Coppertone Sport Sunscreen) state “Water resistance (80 minutes)”. Water resistance was evaluated by measuring SPF performance (using the same method as disclosed in example 14) after initial application of the formulation to a test sample and after immersion of the test samples in water, and comparing the results.

Water Immersion Test and Post-Immersion SPF

The following test was used to determine the percent of an SPF rating for the sunscreen composition that was lost following immersion in water for 80 minutes. When the terms “post-immersion SPF” and “initial SPF” are used herein, the post-immersion SPF refers to the SPF of the sunscreen composition after being subjected to the following water immersion test, and the “initial SPF” refers to the SPF of the sunscreen composition prior to immersion in water for 80 minutes, as described herein.

To begin, each sunscreen sample in Table 15 was applied to a hydrated VITRO-SKIN® synthetic skin substrate and the initial SPF of the composition was measured as described in example 14. The sunscreen composition-treated VITRO-SKIN® samples were then transferred to a water bath at the same time at 40° C. with agitation at 200 rpm for 80 minutes. The samples were removed from the water bath after 80 minutes, patted dry, and the post-immersion SPF of the samples was measured as described in example 14. The post-immersion SPF values given herein are the averaged values over the number of data points. The results are shown in Table 15.

TABLE 15
Water Immersion Test and Post-Immersion SPF

| | | | | | % Loss In |
|---|---|---|---|---|---|
| | | | | | SPF after |
| | | | | | water |
| | | Number of | Initial | 80 VWR | Immersion |
| Sample | Formulation | data points | SPF | SPF | test |
| J&J Pure and Free | TiO₂ (4.9 wt %) and | 5 | 56.09 | 8.66 | −85 |
| Baby (SPF 60)* | ZnO (4.7 wt %) | | | | |
| Example 14.1* | 32 wt % UV cocktail + | 14 | 52 | 39 | −26 |
| | 68% SME | | | | |
| Example 14.2 | 32 wt % UV cocktail + | 18 | 89 | 77 | −14 |
| | 66 wt % SME + 2 wt % | | | | |
| | thickener 2.63 | | | | |

*: comparative examples

The soy methyl ester (SME) was from Enviro saver.

It can be concluded from the results shown in the Table 15 that the exemplary thickener 2.63 has better water resistance than the comparative examples.

### Example 16

### Molecular Weight of Polymeric Rheology Modifiers of the Disclosure

Molecular weight was determined by Hydrodynamic Chromatography with multiangle light

scattering detection (MALS). This method is similar to a standard GPC/MALS, except a smaller pore size column is used compared to standard GPC/MALS, which results in all the separation taking place in the interstitial volume of the GPC column.

Samples were prepared by dissolving about 10 mg of sample in about 10 ml of butylated

hydroxytoluene (BHT) stabilized tetrahyrofuran (THF). Some samples were further diluted 10-fold with THF as necessary.

Column: PL-Gel 100A 5 um 30 cm×7.8 mm

Column Temp: 40° C.

Solvent: tetrahydrofuran with 0.1% BHT preservative

Injection: 50 μl or 25 μl

Detection: Wyatt Dawn Heleos 18 angle MALS 633 nm and Wyatt Optilab T-Rex Refractive index detector

TABLE 16
Molecular weight values of polymeric
rheology modifiers of the invention

| Thickener | Mwx 10⁶ (D) |
|---|---|
| 1 | 272 |
| 2.1 | 81.8 |
| 2.2 | 242 |
| 2.3 | 230 |
| 2.4 | 339 |
| 2.7 | 245 |
| 2.25 | 176 |
| 2.26 | 189.9 |
| 2.27 | 450 |
| 2.28 | 318 |
| 2.37 | 273 |
| 2.65 | 85.2 |
| 2.7 | 17.4 |
| 2.73 | 2.5 |
| 2.74 | 397 |

The foregoing examples are presented by way of illustration and not by way of limitation.

Those skilled in the art will understand other examples and embodiments are encompassed within the present invention. The spirit and scope of the present invention are to be limited only by the terms of any claims presented hereunder.

1. A personal care formulation comprising
a dermatologically acceptable organic liquid, water, and
a cross-linked (meth)acrylate copolymer dissolved in said dermatologically acceptable organic liquid,
wherein the organic liquid is thickened by the cross linked (meth)acrylate copolymer, and
wherein the cross linked (meth)acrylate copolymer comprises at least one alkyl (meth)acrylate and at least one of the following monomers:
a bicyclic (meth)acrylate different from the alkyl (meth)acrylate, and
an aromatic vinyl monomer,
wherein said personal care formulation is an emulsion.

2. The personal care formulation of claim 1 wherein said formulation is a water-in-oil emulsion.

3. The personal care formulation of claim 1 further comprising solid particles suspended in said formulation.

4. The personal care formulation of claim 1 further comprising an aqueous thickener.

5. The personal care formulation of claim 4 wherein said formulation is an oil-in-water emulsion.

6. The personal care formulation of claim 1 further comprising a surfactant.

7. The personal care formulation of claim 1 where said formulation is surfactant free.

8. The personal care formulation of claim 1 wherein said formulation is a hair care formulation.

9. The personal care formulation of claim 1 wherein said formulation is a skin care formulation.

10. The personal care formulation of claim 9 wherein said formulation is a sunscreen formulation.

11. The personal care formulation of claim 10 wherein said formulation comprises at least one organic sunscreen active ingredient.

12. The personal care formulation of claim 3 wherein said solid particles are sunscreen active ingredients and said formulation is a sunscreen formulation.
